(19) United States
(12) Patent Application Publication
(10) Pub. No.: US 2026/0301169 A1
(43) Pub. Date: Oct. 1, 2026

(54) SYSTEMS AND METHODS FOR POINT-OF-CARE ASSESSMENT BASED ON PATHOLOGY IMAGE ANALYSIS

(71) Applicants: Isla P. GARRAWAY, Los Angeles, CA (US); UNIVERSITY OF UTAH RESEARCH FOUNDATION, Salt Lake City, UT (US)

(72) Inventors: Isla P. Garraway, Los Angeles, CA (US); Beatrice S. Knudsen, Salt Lake City, UT (US); Tolga Tasdizen, Salt Lake City, UT (US)

(21) Appl. No.: 19/481,006

(22) PCT Filed: May 3, 2024
(86) PCT No.: PCT/US2024/027824

§ 371 Date: Nov. 3, 2025

Related U.S. Application Data
(60) Provisional application No. 63/464,449, filed on May 5, 2023.
(60) Provisional application No. 63/508,056, filed on Jun. 14, 2023.

(51) Int. Cl. G06T 7/00; G06T 7/155; G16H 30/40; G16H 50/20

(52) U.S. Cl. CPC G06T 7/0012; G06T 7/155; G16H 30/40; G16H 50/20; G06T 2207/10056; G06T 2207/20081; G06T 2207/20084; G06T 2207/30024

(57) ABSTRACT

Computing systems, computing devices, computer program products, and computer-implemented methods to assess cancerous bodily tissue samples. The computer-implemented methods include identifying, by a computing device comprising at least one processor, an area within an image of a section of cancerous bodily tissue. The area encompasses cancerous tissue having a grade that is greater than or equal to a threshold grade. The method also includes generating, by the computing device, a feature vector within the area comprising multiple values defining respective targeted histopathologic features of neuroendocrine differentiation. The method further comprises determining a score for the section of cancerous bodily tissue by applying a machine-learning model to the feature vector. The score represents an amount of neuroendocrine differentiation present in the area. The method can be applied by a computing device at point-of-care in order to provide a pathologist or another type of human expert with an assessment of a patient afflicted by cancer.

### CROSS-REFERENCE TO RELATED APPLICATIONS

This application is a non-provisional of and claims the benefit of U.S. Provisional Patent Application No. 63/464,449, filed on May 5, 2023 and U.S. Provisional Patent Application No. 63/508,056, filed on Jun. 14, 2023, the contents of each application being incorporated herein by reference.

### STATEMENT REGARDING FEDERALLY SPONSORED RESEARCH OR DEVELOPMENT

This work was supported by the United States as represented by the Department of Veterans Affairs (Washington, D.C.), and the Federal Government has certain rights in this invention.

### BACKGROUND

Chromosomal instability (CIN) is associated with poor outcomes in patients, but it is an understudied mechanism of aggressive prostate cancer (PCA). Ell type specific mechanisms of CIN cause clonal diversity that generates metastatic and treatment resistant phenotypes. Despite the strong association between CIN and patient outcomes, CIN is currently not used as a biomarker. CIN is defined as gain or loss of whole chromosomes (numerical/whole CIN) or fractions of chromosomes (structural CIN). One way to measure the amount of CIN in cancer is by the percentage of DNA that is amplified, deleted or rearranged. Published studies demonstrate an association of specific CIN subtypes and aggressive tumor behavior (metastatic progression, castration-resistance, cancer death) in PCA. While these studies demonstrate the potential of CIN as a biomarker, multiple questions remain that need to be addressed.

Measuring CIN is difficult, expensive and tissue destructive. Broadly, CIN can be measured by genomic, cytogenetic, or cytometric approaches. Genomic-based CIN measurements, including comparative genomic hybridization and single nucleotide polymorphism arrays are expensive, in particular when used with formalin-fixed and paraffin-embedded tissues, and not part of the routine clinical workup of solid tumors. Next generation sequencing (NGS) is now standard of care for most advanced solid tumors, and CIN can be inferred from bulk DNA sequencing; however, accurate CIN measurements require samples with a high fraction of tumor which can be difficult to impossible to obtain in the many tumor types such as prostate cancer.

Mechanisms causing CIN in PCA are poorly understood. There are several different subtypes of CIN, defined by the length of chromosomal aberrations and distribution throughout the genome. CIN has multiple causes, however it is unclear which of them prevail in PCA. Mechanisms causing structural and numerical changes of chromosomes include a failure of proper chromosome separation during mitosis, nuclear membrane instability, centrosome defects, formation and rupture of micronuclei in the cytoplasm and progression of genomic instability. Identifying underlying mechanisms of CIN in PCA will open new treatment opportunities.

CIN increases the clonal diversity in cancers, which enhance the emergence of drug resistant cancer cell populations. In particular, rapidly proliferating cancers, such as PCA with Rb1 and p53 loss possess high levels of CIN and drug resistance. CIN in circulating PCA tumor cells was associated with poor outcome and lesser response to abiraterone and enzalutamide. In muscle invasive bladder cancer, CIN predicted increased response to cisplatin. In the TNT trial of triple negative breast cancer, patients with high-level amplifications had a greater treatment response and significantly longer progression free survival in the carboplatin arm compared to the docetaxel arm. In PCA, CIN might be used to stratify patients with hormone sensitive disease to androgen deprivation therapy (ADT) or Enzalutamide/Abiraterone androgen signaling inhibitors (ASI). Based on the results of clinical trials in other cancer types, CIN might be used as a biomarker in PCA to stratify patients for Cabazitaxel versus Carboplatin.

CIN suppresses the immune response to cancers. Another potential application of a CIN biomarker is for treatment with immunotherapy drugs. In PCA, CIN might be one reason for the immunosuppressed environment that exists in most PCAs and makes them refractory to checkpoint inhibition.

Accordingly, CIN has the potential for a prognostic and treatment related (predictive) biomarker, and the CIN status may provide a rationale for management of PCA patients who require the most aggressive treatment at the time of diagnosis. It is desirable to provide a system and method that uses digital pathology and image analysis with machine learning/A.I. algorithms to develop an assay for CIN that is affordable, easy to deploy and that can be used with regular pathology slides which are available from patients with a diagnosis of PCA.

### SUMMARY

It is to be understood that both the following general description and the following detailed description are illustrative and explanatory only and are not restrictive.

The present disclosure describes an image-based machine learning pipeline that provides a measurement of cell plasticity, including chromosomal instability (CIN) in tissue samples (e.g., human, animal, plant). Also described is a companion diagnostic for CIN positive cancers using computational histopathology and quantitative immunohistochemistry. This companion diagnostic integrates data from multiple domains using artificial intelligence.

In contrast to other approaches, this disclosure describes use of individual tumor cells and nuclei and force algorithms to learn on nuclear and peri-nuclear features as well as on data from multiple domains (e.g., Hematoxylin (H) and Eosin (E) (H&E), IHC, RNA) that are known to be associated with CIN. The main differences to other published approaches include: not focusing on dividing cells but on the detection of CIN in non-mitotic cancer cells and therefore applicable to cancers with low proliferation rates, precancerous conditions and a broader range of different cancer types; detection of multifactorial, molecular causes of CIN and not simply on the presence of atypical mitoses. This approach is broader than just CIN and includes the concept of cell plasticity, which in prostate cancer includes CIN, neuroendocrine differentiation and epithelial to mesenchymal transition (EMT).

In one embodiment, the disclosure provides a computer-implemented method. The computer-implemented method includes identifying, by a computing device comprising at least one processor, an area within an image of a section of cancerous bodily tissue. The area encompasses cancerous tissue having a grade that is greater than or equal to a threshold grade. The method also includes generating, by the computing device, a feature vector within the area comprising multiple values defining respective targeted histopathologic features of neuroendocrine differentiation. The method further comprises determining a score for the section of cancerous bodily tissue by applying a machine-learning model to the feature vector. The score represents an amount of neuroendocrine differentiation present in the area. The method can be applied by a computing device at point of care in order to provide a pathologist or another type of human expert with an assessment of a patient afflicted by cancer.

In another embodiment, the disclosure provides a computer-implemented method for pathology image classification. The method includes receiving an image of a cancerous tissue sample for a patient, generating a feature vector within an area on the image, wherein the feature vector includes multiple values defining respective targeted histopathologic features of neuroendocrine differentiation, determining a metric for the area of cancerous tissue sample on the image by processing the feature vector through a model trained to distinguish tiles of non-metastatic (M0) adenocarcinoma from tiles of small cell carcinoma (SC), and wherein the metric represents an amount of neuroendocrine differentiation present in the area, and providing the metric for further processing or to a user interface of a computer device.

The disclosure also provides computing systems, computing devices, and computer program products that, at least, can implement one or more of the disclosed computer-implemented methods.

Additional elements or advantages of this disclosure will be set forth in part in the description which follows, and in part will be apparent from the description, or may be learned by practice of the subject disclosure. The advantages of the subject disclosure can be attained by means of the elements and combinations particularly pointed out in the appended claims.

This summary is not intended to identify critical or essential features of the disclosure, but merely to summarize certain features and variations thereof. Other details and features will be described in the sections that follow. Further, both the foregoing general description and the following detailed description are illustrative and explanatory only and are not restrictive of the embodiments of this disclosure.

Other aspects of the disclosure will become apparent by consideration of the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The annexed drawings are an integral part of the disclosure and are incorporated into the subject specification. The drawings illustrate example embodiments of the disclosure and, in conjunction with the description and claims, serve to explain at least in part various principles, elements, or aspects of the disclosure. Embodiments of the disclosure are described more fully below with reference to the annexed drawings. However, various elements of the disclosure can be implemented in many different forms and should not be construed as limited to the implementations set forth herein. Like numbers refer to like elements throughout.

FIG. 1 illustrates an example of an operating environment for computer-assisted point of care based on pathology image analysis, in accordance with one or more embodiments of this disclosure.

FIG. 2 illustrates examples of targeted descriptors used to train models for pathology image analysis, in accordance with one or more embodiments of this disclosure.

FIG. 3A illustrates results from a machine-learning model that yields scores indicative of neuroendocrine differentiation (NED) in cancerous bodily tissue, in accordance with one or more embodiments of this disclosure.

FIG. 3B illustrates results from a model that predicts a score indicative of metastasis risk (MR) in cancerous bodily tissue, in accordance with one or more embodiments of this disclosure.

FIG. 3C is a Venn diagram that illustrates a number of features shared by a model that predicts an MR score and another model that predicts a NED score, in accordance with one or more embodiments of this disclosure.

FIG. 4A illustrates another example of an operating environment for computer-assisted point of care based on pathology image analysis, in accordance with one or more embodiments of this disclosure.

FIG. 4B illustrates an example of perinuclear rings about a cancerous cell nucleus, and example filter operations that can be applied to determined select targeted features that correlate to chromosomal instability (CIN) scores, in accordance with one or more embodiments of this disclosure.

FIG. 4C illustrates notched boxplots for nuclear morphology features correlated to CIN scores, in accordance with one or more embodiments of this disclosure.

FIG. 5 illustrates an example of a method for developing a model to quantify neuroendocrine differentiation in pathology images of high-grade prostate cancer, in accordance with one or more embodiments of this disclosure.

FIG. 6 illustrates an example of a method for selecting features to define a feature vector that can be used to determine a NED score, in accordance with one or more embodiments of this disclosure.

FIG. 7 illustrates an example of a method for assessing an extent of neuroendocrine differentiation in a section of cancerous bodily tissue, in accordance with one or more embodiments of this disclosure.

FIG. 8 illustrates an example of a method for assessing an extent of neuroendocrine differentiation in a section of cancerous bodily tissue, in accordance with one or more embodiments of this disclosure.

FIG. 9 illustrates an example of a method for assessing prognosis of a high-grade cancer, in accordance with one or more embodiments of this disclosure.

FIG. 10 illustrates an example of a computing system to implement aspects of computer-assisted point of care based on pathology image analysis, in accordance with one or more embodiments of the disclosure.

FIG. 11 illustrates an example of a method for analyzing pathology slides where an H&E RGB image is separated into H and E channels and processed separately to generate two feature sets f_{H} and f_{E} trained with the proposed contrastive loss.

FIG. 12 illustrates examples of ccRCC and prostate gland tiles.

### DETAILED DESCRIPTION

One or more embodiments are described and illustrated in the following description and accompanying drawings. These embodiments are not limited to the specific details provided herein and may be modified in various ways. Furthermore, other embodiments may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way but may also be configured in ways that are not listed. Furthermore, some embodiments described herein may include one or more electronic processors configured to perform the described functionality by executing instructions stored in a non-transitory, computer-readable medium. Similarly, embodiments described herein may be implemented as non-transitory, computer-readable mediums storing instructions executable by one or more electronic processors to perform the described functionality. As used in the present application, “non-transitory computer-readable medium” comprises all computer-readable media but does not consist of a transitory, propagating signal. Accordingly, a non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

In addition, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms such as first and second, top and bottom, and the like may be used herein solely to distinguish one entity or action from another entity or action without necessarily requiring or implying any actual such relationship or order between such entities or actions.

Unless the context of their usage unambiguously indicates otherwise, the articles “a,” “an,” and “the” should not be interpreted as meaning “one” or “only one.” Rather these articles should be interpreted as meaning “at least one” or “one or more.” Likewise, when the terms “the” or “said” are used to refer to a noun previously introduced by the indefinite article “a” or “an,” “the” and “said” mean “at least one” or “one or more” unless the usage unambiguously indicates otherwise.

It should also be understood that although certain drawings illustrate hardware and software located within particular devices, these depictions are for illustrative purposes only. In some embodiments, the illustrated components may be combined or divided into separate software, firmware, and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable communication links.

Thus, in the claims, if an apparatus or system is claimed, for example, as including an electronic processor or other element configured in a certain manner, for example, to make multiple determinations, the claim or claim element should be interpreted as meaning one or more electronic processors (or other element) where any one of the one or more electronic processors (or other element) is configured as claimed, for example, to make some or all of the multiple determinations. To reiterate, those electronic processors and processing may be distributed.

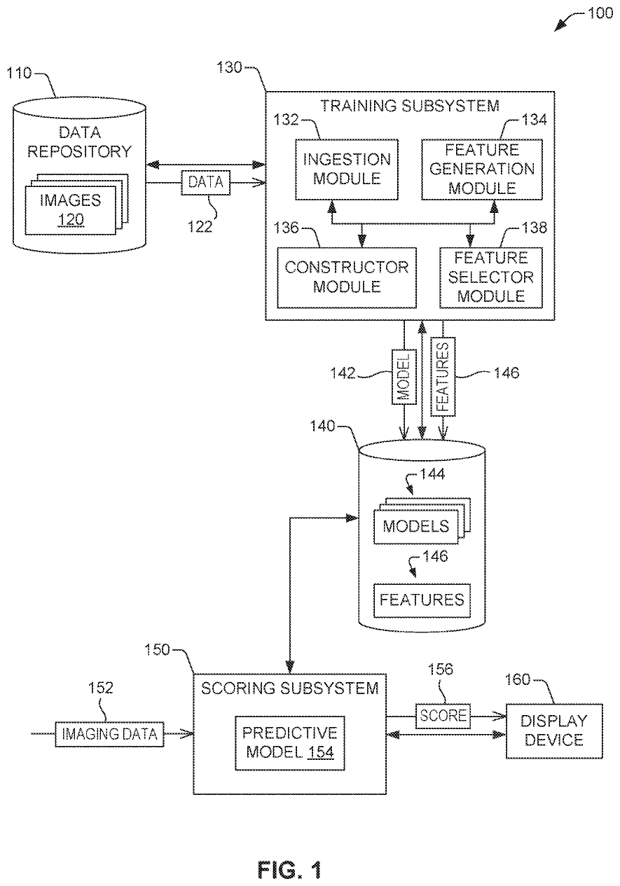

FIG. 1 illustrates an example of an operating environment 100 for computer-assisted point of care based on pathology image analysis, in accordance with one or more embodiments of this disclosure. The operating environment 100 can include a data repository 110 that can retain multiple images 120. A subset of the multiple images 120 can include digital slides. For example, the digital slides can depict cancerous bodily tissue having adenocarcinoma of prostate. The disclosure, however, is not limited in this respect, and adenocarcinoma of other organs also is contemplated. Such digital slides can be regular pathology slides for microscopic diagnosis. Imaging equipment that probes photoluminescence of cancerous bodily tissue stained with a dye can generate the digital slides. In one example, the dye is a mixture of hematoxylin and eosin. A second subset of the multiple images 120 can include other digital slides, each depicting cancerous bodily tissue having small cell carcinoma. The tissue depicted in those other digital slides can be lung tissue and prostate tissue, for example.

The operating environment 100 also can include a training subsystem 130. As is illustrated in FIG. 1, the training subsystem 130 can include an ingestion module 132 that can receive various types of data. Thus, the ingestion module 132 can receive imaging data 122 from the data repository 110. The imaging data 122 can include first imaging data representing multiple digital slides depicting respective sections of cancerous bodily tissue including adenocarcinoma of prostate. Those sections of cancerous bodily tissue can be extracted from primary tumors, without any sections obtained from biopsies of metastatic sites. The ingestion module 132 can operate on at least a subset of the multiple digital slides to partition each digital slide into multiple tile images (or tiles), thus generating a group of first tile images corresponding to the cancerous bodily tissue. Each one of the first tile images can depict cancerous tissue having a grade equal to or greater than a threshold grade. For the example, the grade can be one of the grade groups in the Gleason scale and the threshold grade can be grade group 4 in the Gleason scale.

The imaging data 122 also can include second imaging data representing multiple digital slides depicting respective sections of other cancerous bodily tissue including small cell carcinoma. The ingestion module 132 also can operate on at least a subset of those multiple digital slides to partition each digital slide into multiple tile images, thus generating a group of second tile images corresponding to that other cancerous bodily tissue. Such sections of cancerous bodily tissue also can be extracted from primary tumors, without any sections obtained from biopsies of metastatic sites.

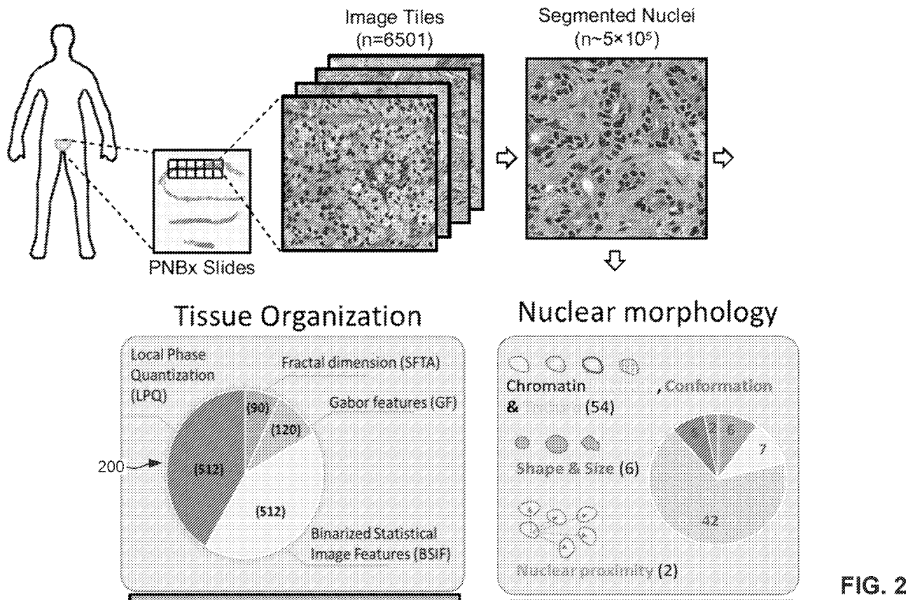

The training subsystem 130 also includes a feature generation module 134 that can operate on the group of first tile images and the group of second tile images. More specifically, the feature generation module 134 can generate first feature vectors for respective tile images of the group of first tile images. Each one of the first feature vectors is defined in terms of a set of targeted descriptors (or targeted features) individually indicative of tissue organization. Thus, the components of a feature vector correspond to respective descriptors in the set of targeted descriptors. The set of targeted descriptors has n items, each pertaining to one of several descriptor categories. Those categories include, for example, fractal dimensions, Gabor features, binarized statistical image features, and local phase quantization (LPQ) features. Thus, each one of the first feature vectors is an n-dimensional feature vector. Accordingly, generating a feature vector includes determining n values defining respective tissue organization descriptors. Tissue organization descriptors can be embodied in histopathological features of neuroendocrine differentiation. In other cases, histopathological features of other forms of lineage plasticity also can be contemplated. In some cases, n can be greater than 1000. In one example, as is shown in FIG. 2, n is equal to 1234 and those n features can be distributed across four descriptor categories according to the distribution shown in pie chart 200. This disclosure is of course not limited in that respect, and other distributions across targeted descriptor categories can be contemplated. For example, other targeted features include size features, shape features, and distance features. In some cases, a targeted feature can be a handcrafted feature. Handcrafted features are features defined empirically or according to one or more heuristics.

Further, the feature generation module 134 also can generate second feature vectors for respective tiles images of the group of second tile images. Each one of the second feature vectors is defined in terms of the same set of targeted descriptors (or targeted features) used to define the first feature vectors. Thus, the components of a feature vector of the second feature vectors correspond to respective descriptors in the set of targeted descriptors. Hence, each one of the second feature vectors also is an n-dimensional feature vector. Accordingly, generating each feature vector of the second feature vectors includes generating n values defining respective tissue organization descriptors. Again, in one example, n is equal to 1234 and those n features can be distributed across four descriptor categories according to the distribution shown in pie chart 200 (FIG. 2). It is noted that descriptor categories shown in that pie chart 200 can be referred to as texture features.

The training subsystem 130 also includes a constructor module 136 that can train multiple machine-learning models. Each one of the multiple machine-learning models can be trained to distinguish tiles of non-metastatic (M0) adenocarcinoma from tiles of small cell (SC) carcinoma. Thus, each one of those trained machine-learning models can be referred to as a M0/SC model. Each machine-learning model of the multiple machine-learning models can be trained using the first feature vectors and the second feature vectors. Before training, individual features having respective values that are highly correlated or invariant can be excluded. That is, features having values within a small range and similar to each other can be excluded before training. To train the multiple machine-learning models, the constructor module 136 can implement a bootstrapping approach or a cross-validation approach, or both. Any cross-validation approach can be used. Each one of the machine-learning models can be embodied in one of a support vector machine (SVM); a random forest (RF) model; a multi-layer perceptron; lasso and elastic-net regularized generalized linear models (GLMNET); or similar model.

After training a machine-learning model 142, the constructor module 136 can retain the trained machine-learning model 142 within one or more memory devices 140 (which can be referred to as data storage 140). The trained machine-learning model 142 can be retained as part of a group of multiple machine-learning models 144. Such a group can be configured within a database containing machine-learning models or a filesystem that contains files defining machine-learning models.

The machine-learning models that the constructor module 136 has trained using the first feature vectors and the second feature vectors can be used to select a subset of the set of targeted descriptors. Such a subset can define a select feature vector that has m components (m<n, for example) each component corresponding to a select targeted descriptor. The feature vector is common to all machine-learning models that the constructor module 136 has trained.

The training subsystem 130 can include a feature selector module 138 that can select the subset of the set of targeted descriptors. To that end, the feature selector module 138 can obtain a machine-learning model. The machine-learning model can be obtained from the group of multiple machine-learning models 144. The feature selector module 138 can then generate, using the obtained machine-learning model, a ranking of targeted descriptors according to variable/feature importance. The ranking can be embodied in a list of targeted descriptors arranged in decreasing order of importance. To determine the importance of a variable, the feature selector model 138 can determine values of an importance metric (Gini impurity, for example) applicable to the machine-learning model, after the machine-learning model has been trained. Thus, each target descriptor in the set of target descriptors can be ascribed a value representative of weight of the target descriptor in a prediction of the machine-learning model. The validation sample can include multiple features vectors evaluated using a group of image tiles designated for model validation.

The feature selector module 138 can obtain another machine-learning model. That other machine-learning model also can be a trained machine-learning model from the group of multiple machine-learning models 144. The feature selector module 138 can then generate, using that other machine-learning model, a second ranking of targeted descriptors according to variable/feature importance. The second ranking can be embodied in a list of targeted descriptors arranged in decreasing order of importance.

The feature selector module 138 can continue generating rankings of targeted descriptors according to variable/feature importance for other machine-learning models until a termination criterion is satisfied. For example, the termination criterion can dictate that all models within a preset group of machine-learning models must be analyzed to determine respective rankings of targeted descriptors.

After the termination criterion has been satisfied, the feature selector module 138 can aggregate the generated rankings of targeted descriptors, where each one of the generated rankings correspond to respective machine-learning models. Aggregation of such rankings results in an aggregated ranking of targeted descriptors according to variable/feature importance. The feature selector module 138 can then determine the select feature vector based on such an aggregated ranking. Specifically, the feature selector module 138 can select a subset of the set of targeted descriptors, where each targeted descriptor in the subset has a variable/feature importance that meets or exceeds a threshold value. The feature selector module 138 can assign the select targeted descriptors 146 in that subset to respective components of the select feature vector. The feature selector module 138 can retain the select targeted descriptors 146 in the data storage 140. The select targeted descriptors 146 can be retained within a configuration file, for example. The select feature vector is common to all machine-learning models used to generate aggregated ranking.

The select feature vector can be used with a particular trained machine-learning model to determine a score indicative of neuroendocrine differentiation in an image of a section of cancerous bodily tissue having high-grade cancer (e.g., Gleason grade group equal to or greater than 4). That score can be referred to as NED score. The NED score can be a real number (range 0-1, for example) indicative of how much the cancerous bodily tissue with adenocarcinoma histology “looks like” cancerous bodily tissue with small cell carcinoma. More specifically, the select feature vector can be evaluated for multiple tile images originating from the image of the section of cancerous bodily tissue. In addition, the particular trained-machine learning model can be applied to the evaluated select feature vector, yielding the NED score for the section of cancerous bodily tissue.

As is shown in FIG. 1, the operating environment 100 includes a scoring subsystem 150 that can determine NED scores using a predictive model 154. The predictive mode 154 is M0/SC model. The scoring subsystem 150 can obtain the predictive model 154 from the group of machine-learning models 144 within the model storage 140.

The scoring subsystem 150 can receive imaging data 152 defining an image (e.g., a digital slide) of a section of cancerous bodily tissue with high-grade cancer. The scoring subsystem 152 can identify a tile within the image, where the tile depicts an area encompassing cancerous tissue having a cancer grade that is equal to or greater than a threshold grade. To identify the tile, the scoring subsystem 150 can apply a cancer grading algorithm to the image, resulting in the grade, and can then compare the resulting grade to the threshold grade. Simply as an example, the cancer grade can be one of the grade groups in the Gleason scale and the threshold grade can be grade group 4 in the Gleason scale.

The scoring subsystem 150 can access configuration data defining the select feature vector containing the select targeted descriptors 146. The configuration data can be accessed from the data storage 140, in some cases. The scoring subsystem 150 can generate, based on the tile, an instance of the select feature vector. Generating that instance of the select feature vector includes generating multiple values defining the tissue organization features. Those tissue organization features are select targeted descriptors, each representing histopathological features of neuroendocrine differentiation. As is described herein, each one of the select targeted descriptors satisfies a variable/feature importance criterion.

The scoring subsystem 150 can determine a NED score 156 for the section of the cancerous bodily tissue by applying the predictive model 154 to the instance of the select feature vector comprising the select targeted descriptors 146. The NED score 156 quantifies neuroendocrine differentiation present in that section. The NED score 156 determined for a single tile can be ascribed to the image (e.g., digital slide) of the section of cancerous bodily tissue with high-grade cancer. In other cases, the scoring subsystem 150 can identify multiple tiles of the image and can determine respective NED scores for the multiple tiles. The respective NED scores can be determined by applying the predictive model 154 to respective instances of the selected feature vector. That is, for each one of the multiple tiles, the scoring subsystem 150 can determine an instance of the select feature vector, and can then apply the predictive model 154 to each of those instances, resulting in the respective NED scores. The scoring subsystem 150 can then determine an overall NED score for the image. To that end, the scoring subsystem 150 can apply an ensemble method to aggregate the respective NED scores for each tile and, thus, generate the overall NED score. The ensemble method can include, in some cases, using the mean of all NED scores across tiles.

The scoring subsystem 150 can cause presentation of the NED score 156. To that end, the scoring subsystem 150 can cause a display device 160 to present the NED score 156, for example. Causing the display device 160 to present the score can include directing the display device 160 to present a visual element indicative of the NED score 156. The visual element can include, for example, text and/or an image indicative of the NED score 156. The visual element can be part of a user interface presented by the display device. The display device 160 can be integrated into a computing device that embodies, or hosts, the scoring subsystem 150. In other cases, the display device 160 can be functionally coupled, via a wireless or wireline connection, for example, to the computing device. In addition, or in other embodiments, the scoring subsystem 150 can embed the NED score 156 in a pathology report. The scoring subsystem 150 can send the pathology report to an electronic address (e.g., an email address or a mobile telephone number) of a pathologist. The scoring subsystem 150 also can cause a printer device (not depicted in FIG. 1) to output the pathology report including the NED score 156.

In some embodiments, a computing device can host the scoring subsystem 150. The computing device can be deployed at point of care, which can be remotely located from the training subsystem 130. Thus, the computing device can provide a pathologist or another type of human expert with NED scores at point of care, thus simplifying the diagnostics stage for a patient. By hosting the scoring subsystem 150 at point-of-care, the computing device can determine the stage of the patient's cancer and/or the prognosis of the patient. That is, the computing device can determine what risk the patient has that the cancer progresses to a lethal form that is difficult to treat. Access of such information at point-of-care can permit readily applying treatment consistent with that risk and/or prognosis.

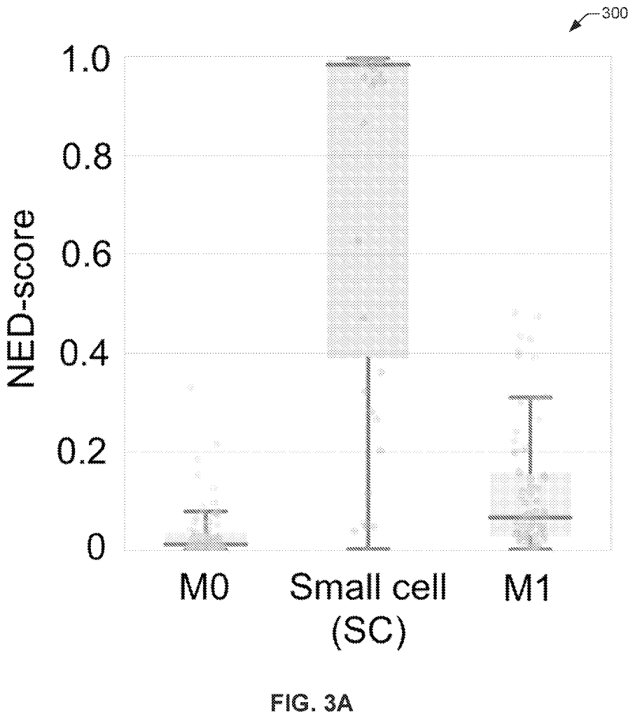

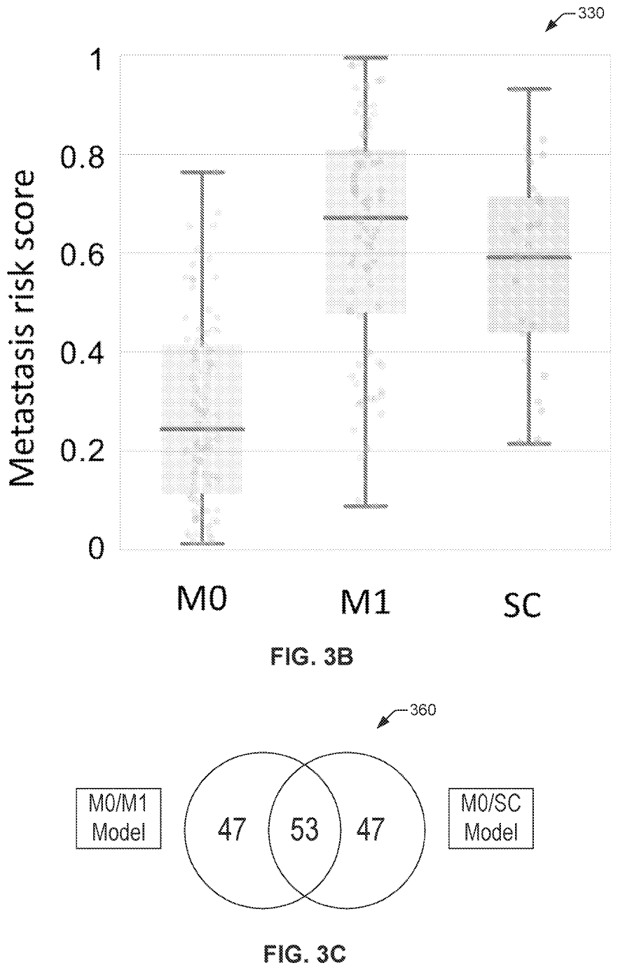

FIG. 3A illustrates results from an example of the predictive model 154, which model predicts NED scores in cancerous bodily tissue having high-grade cancer, in accordance with one or more embodiments of this disclosure. The results are presented in boxplots in a single graph 300. The results show that tiles from a primary tumor that has metastasized (referred to as M1 cases; denoted by “M1” in the abscissa of graph 300) are more “small cell-like” than other tiles from non-metastatic cancerous bodily tissue (referred to as M0 cases; denoted by “M0” in the abscissa of graph 300). NED scores of histologically confirmed small cell cancers are greater than NED scores of M1 adenocarcinomas cases. In some cases, all tiles are from a primary prostate biopsy and not from metastatic sites.

As a comparison, FIG. 3B illustrates results from a model that predicts a score indicative of metastasis risk (MR) in cancerous bodily tissue, in accordance with one or more embodiments of this disclosure. That score can be referred to as MR score. The model can be a machine-learning model trained to distinguish tiles of non-metastatic prostate adenocarcinoma (M0) from tiles of metastatic adenocarcinoma (M1). Thus, the model can be referred to as a M0/M1 model. In some cases, all tiles can be obtained from a primary tumor and not from metastatic sites. To determine an overall MR score of a section of cancerous bodily tissue, the trained model can be applied to small cell (SC) tiles, and the resulting MR scores of respective tiles can be averaged to yield the overall MR score. The/e MR score can be calculated using a leave-one-out approach, in some cases.

Analysis of the select targeted descriptor (or features) that are relied upon to train the M0/SC model (which yields a NED score) and M0/M1 model (which yields an MR score) reveals that those models share features that cannot be seen by a human pathologist. The shared features are readily detected by a computing device applying either one of those models. Additionally, the shared features constitute a significant proportion of targeted feature relied upon by each model in the prediction of a score (NED score or MR score).

FIG. 3C is a Venn diagram 360 that illustrates a number of features shared by the M0/M1 model and the M0/SC model. As is described herein, bootstrapping and cross-validation can be used to train multiple machine-learning models (e.g., the group of machine-learning models 144 (FIG. 1)). In one example, multiple first random-forest (RF) models can be trained using approximately 1,300 tissue organization features to distinguish M0 tiles from SC tiles. In addition, multiple second RF models can be trained using the approximately 1,300 tissue organization features to distinguish or M0 tiles from M1 tiles. After training, for each one of those models, the top 100 features can be identified in a ranking according to feature importance. Overlap of features between the M0/SC model and the M0/M1 model indicates that 53 features out of the 100 features are shared between the M0/SC model and the M0/M1 model.

At least some features obtained from images of sections of cancerous bodily tissue can be correlated to chromosomal instability (CIN) scores. Correlated features can be referred to as CIN-F features and can predict prognosis in patients with high-grade prostate cancer, for example. CIN-F features also can be referred to as CIN-F descriptors.

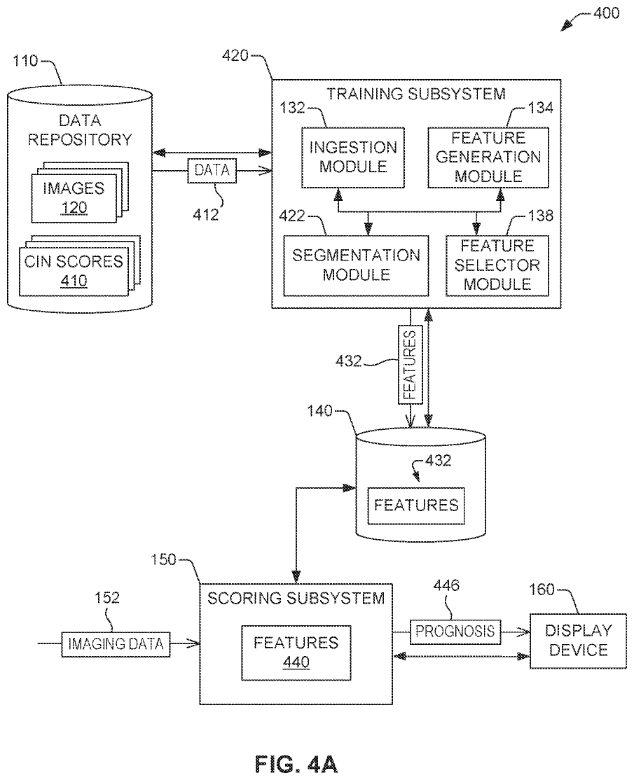

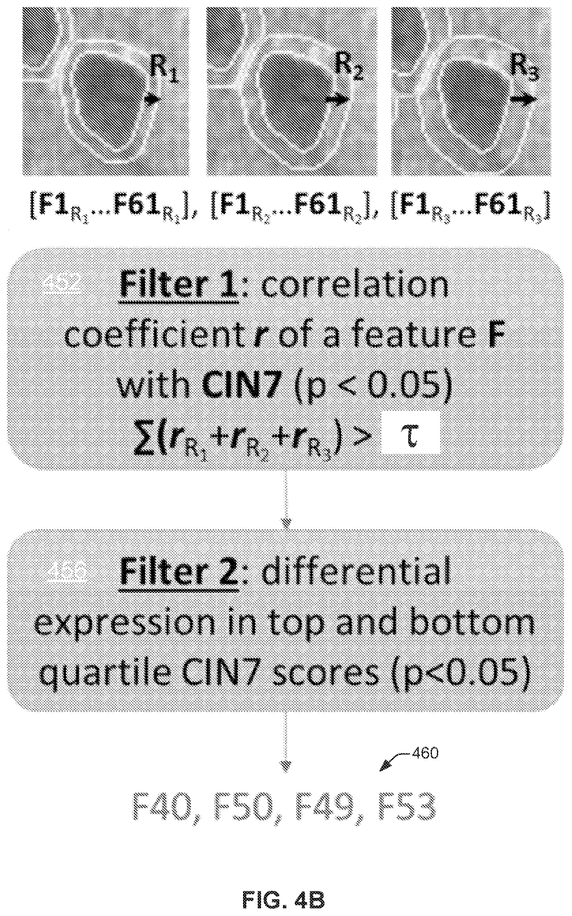

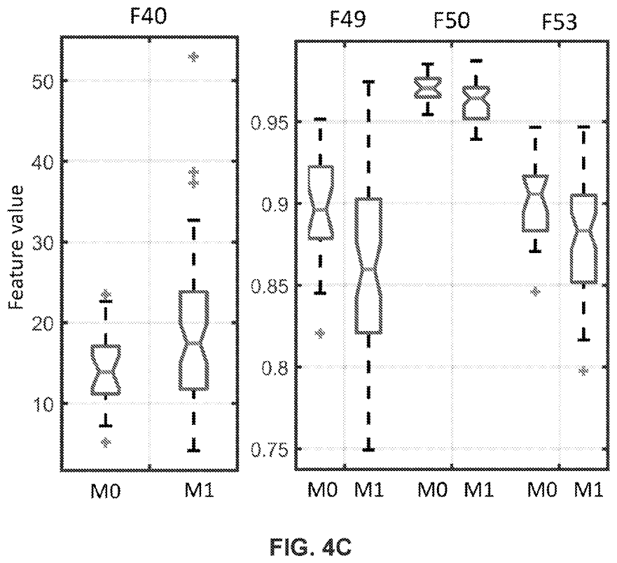

FIG. 4A illustrates an operating environment 400 for computer-assisted point-of-care based on pathology image analysis, where CIN-F features can be determined and applied in the pathology image analysis, in accordance with one or more embodiments of this disclosure. As is illustrated in FIG. 4A, a training subsystem 420 can obtain at least some of the images 120 depicting sections of cancerous bodily tissue. To that end, the ingestion module 132 can receive data 412. The data 412 can include imaging data defining multiple digital slides depicting respective sections of cancerous bodily tissue that has adenocarcinoma (e.g., prostate adenocarcinoma). The ingestion module 132 can operate on at least a subset of the multiple digital slides to partition each digital slide into multiple tile images, thus generating a group of first tile images corresponding to the cancerous bodily tissue. Each one of the first tile images can depict cancerous tissue having a grade equal to or greater than a threshold grade. The data 412 also can include CIN scores corresponding to respective sections of the cancerous bodily tissue. Each one of the CIN scores can be, for example, a score determined based on seven CIN genes identified using ribonucleic acid (RNA) sequencing data of sections of cancerous bodily tissue having a grade that is equal to or greater than the threshold grade. Such a score can be referred to as a CIN-7 score. For example, both cancer grade of cancerous tissue depicted by first tile images and cancer grade of cancer tissue assessed using CIN-7 scores can be quantified in terms grade groups in the Gleason scale. The threshold grade can be grade group 4 in the Gleason scale.

The training subsystem 420 also includes a segmentation module 422 that can identify a one or more cancer nuclei within each tile image of the group of first tile images. To that end, the segmentation module 422 can apply a nuclear segmentation model to datasets of the imaging data defining the group of first tile images. Each one of the datasets defines a tile image of the group of first tiles images. In some cases, the nuclear segmentation model can be embodied in a mask regions-with-CNN-features (R-CNN) model.

The segmentation module 422 can determine multiple annular regions about a cancer nucleus identified within a tile image of the group of first tile images. Each one of the multiples annular regions includes a periphery of the cancer nucleus. Thus, each annular region can be referred to as a perinuclear ring and corresponds to the perinuclear space of a nucleus. In some cases, as is shown in FIG. 4B, the segmentation module 422 can determine three annular regions R1, R2, and R3 can be determined. In some configurations, the segmentation module 422 determines multiple annular regions about each one of the cancer nuclei identified within the tile image.

The training subsystem 420 also includes the feature generation module 134. The feature generation module 134 can determine nuclear morphology features (or descriptors) for the multiple annular regions (e.g., R1, R2, and R3 (FIG. 4B)). More specifically, the feature generation module 134 can determine a set of targeted nuclear morphology features can be determined for each annular region. The set of targeted nuclear morphology features has m items, each pertaining to one of various descriptor categories that characterize morphology of a cell nucleus. The descriptor categories include chromatin intensity, chromatin conformation, chromatin texture, shape, size, and nuclear proximity. That set of targeted nuclear morphology features can have m items: F1, F2 . . . , Fm. Thus, an m-dimensional feature vector can be generated for each annular region, where the components of that feature vector correspond to respective descriptors in the set of targeted nuclear morphology features. Accordingly, generating a feature vector includes determining in values defining respective tissue organization descriptors. In one example, as is shown in FIG. 2, m can be equal to 62 and those m features can be distributed across five descriptor categories according to the distribution shown in pie chart 250. This disclosure is of course not limited in that respect, and other distributions across descriptor categories can be contemplated. For example, generating the feature vector can include determining values of m=61 texture features.

The training subsystem also includes the feature selector module 138. The feature selector module 138 can determine a subset 432 of the nuclear morphology features that correlate with the CIN-7 scores. Features in that subset are CIN-F features, and the subset 432 includes k CIN-F features. In addition, each one of the k CIN-F features are differentially expressed in top and bottom quartile CIN-7 scores. The feature selector module 138 can retain the subset 432 within the data storage 140. The subset 432 can be retained within a configuration file, for example, containing definitions of respective CIN-F features in the subset 432.

To determine the k CIN-F features, the feature selector module 138 can apply two filters to the set {F1, F2 . . . , Fm}. As is shown in FIG. 4B, applying a first filter 452 of the two filters can include determining, for each feature Fj (j=1, 2 . . . , m), a correlation coefficient

$${r}_{ρ}^{( j )}$$

of the feature Fj with CIN-7 scores included in the data 412. Here, ρ identifies one of the multiple annular regions that have been determined for a cell nucleus. For example, ρ can be one of R1, R2, or R3. Applying the first filter 452 also includes determining, for each feature Fj, the sum of correlation coefficients over the multiple annular regions. That sum can be represented by Σⱼ. Further applying the first filter 452 can then include selecting feature(s) having Σⱼ greater than a threshold value τ (e.g., 0.85, 0.90, or 0.95). In cases where annular regions R1, R2, or R3 are determined,

$${\sum }_{j} = {r}_{ρ}^{( j )} + {r}_{ρ}^{( j )} + {r}_{ρ}^{( j )} .$$

By applying the first filter 452, the feature selector module 138 identifies candidate nuclear morphology features that are candidates for CIN-F features. The feature selector module 138 can thus apply a second filter 456 to select candidate features that are differentially expressed in both high-CIN-7 subgroup and low-CIN-7 subgroup of the CIN scores included in the data 412. Here, differentially expressed means that the expression of the feature is significantly different between the M0 cases and the M1 cases. In some cases, the high-CIN-7 subgroup contains scores in the third quartile (top 25% scores), and the low-CIN-7 subgroup contains scores in the first quartile (bottom 25% scores). Each one of the selected candidate features is a CIN-F features. The group of select features 460 constitutes an example of a group of CIN-F features. The group of select features 460 can include texture features, such as individual perinuclear features F40, F49, F50, and F53.

FIG. 4C illustrates notched boxplots for respective example CIN-F, in accordance with one or more embodiments of this disclosure. Each example CIN-F feature separates high-CIN-7 score (third quartile Q1) and low-CIN-7 score (first quartile Q1), with p-value less than 0.01. Each notched boxplot illustrates, for a particular CIN-F feature, the difference in expression in non-metastatic (M0) high-grade prostate cancer cases and metastatic (M1) high-grade prostate cancer cases at diagnosis. Here, M0 can indicate cases with no metastatic progression for 5 years after diagnosis, and M1 can indicate cases diagnosed with metastatic disease.

With further reference to FIG. 4A, the scoring subsystem 150 can obtain configuration data that defines select CIN-7 features 440. The configuration data can be accessed from the data storage 140, in some cases. As mentioned, the scoring subsystem 150 can receive imaging data 152 defining an image of a section of cancerous bodily tissue with high-grade cancer. The scoring subsystem 152 can determine a tile within the image, where the tile depicts an area encompassing cancerous tissue having a cancer grade that is equal to or greater than a threshold grade (e.g., grade group 4 in the Gleason scale).

The scoring subsystem 150 can then generate, based on the tile, an instance of the select feature vector. Generating that instance of the select CIN-7 features 440 includes determining multiple values defining respective ones of the CIN-7 features 440. Such values, individually or in combination, can predict prognosis in patients with high-grade prostate cancer. Each one of respective values of the CIN-7 features 440 can be normalized to a value in a range from zero to 1, for example. In some cases, the scoring subsystem 150 can determine, using the instance of the CIN-7 features 440, a prognosis metric 446 indicative of prognosis in a patient with high-grade prostate cancer. Because each one of the CIN-7 features 440 is indicative of such a prognosis, the prognosis metric 446 can be one of the CIN-7 features 440 in that instance. In other cases, the prognosis metric 446 can be a combination of at least one CIN-7 and NED score and/or other clinical variables into a nomogram to predict cancer progression risk.

The scoring subsystem 150 can cause presentation of the prognosis metric 446. To that end, the scoring subsystem 150 can cause the display device 160 to present the score, for example. Causing the display device 160 to present the prognosis metric 446 can include directing the display device 160 to present one or multiple visual elements indicative of the prognosis metric 446. The visual element can include, for example, text and/or image(s) indicative of the prognosis metric 446. The visual element can be part of a user interface presented by the display device 160. In addition, or in other embodiments, the scoring subsystem 150 can embed the prognosis metric 446 in a pathology report. The scoring subsystem 150 can then send the pathology report to an electronic address (e.g., an email address or a mobile telephone number) of a pathologist. The scoring subsystem 150 also can cause a printer device (not depicted in FIG. 4A) to output the pathology report including the prognosis metric 446.

As mentioned, in some embodiments, a computing device can host the scoring subsystem 150 and can be deployed at point of care. Thus, the computing device can provide a pathologist or another type of human expert with prognosis metrics at point of care, thus simplifying the diagnostics stage for a patient.

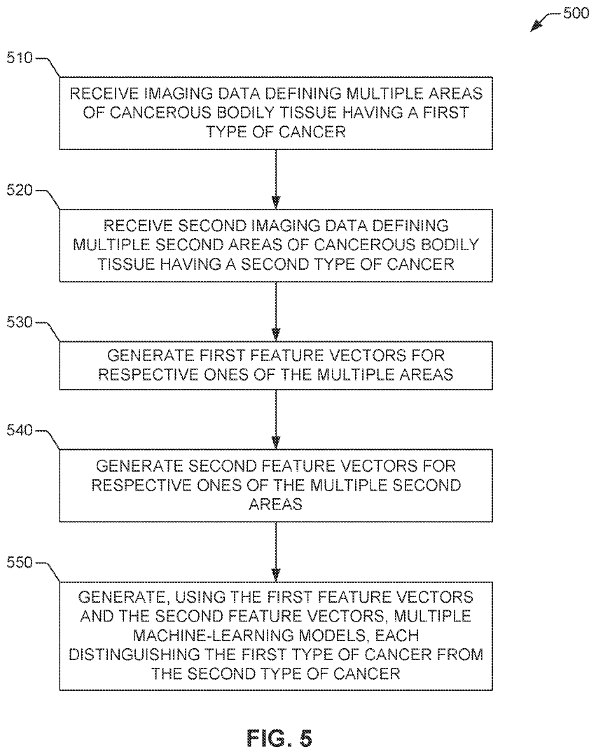

FIG. 5 illustrates an example of a method 500 for generating machine-learning models to distinguish a first type of cancer from a second type of cancer, in accordance with one or more embodiments of this disclosure. For example, the first type of cancer can be adenocarcinoma and the second type of cancer can be small cell carcinoma. A computing device or a system of computing devices may implement the example method 500 in its entirety or in part. To that end, each one of the computing devices includes computing resources that may implement at least one of the blocks included in the example method 500. The computing resources can include, for example, central processing units (CPUs), graphics processing units (GPUs), tensor processing units (TPUs), memory, disk space, incoming bandwidth, and/or outgoing bandwidth, interface(s) (such as I/O interfaces or APIs, or both); controller devices(s); power supplies; a combination of the foregoing; and/or similar resources. The computing resources also can include, for example, programming interface(s); an operating system; software for configuration and/or control of a virtualized environment; firmware; and similar resources. As an example, each of one or multiple ones of the computing devices can embody, or can host, the training subsystem 130 (FIG. 1).

At block 510, the computing device can receive imaging data representing multiple areas of cancerous bodily tissue having the first type of cancer. Each area of the multiple areas can encompass cancerous tissue having a grade equal to or greater than a threshold grade. For the example, the grade can be one of the grade groups in the Gleason scale and the threshold grade can be grade group 4 in the Gleason scale. The multiple areas can correspond to tiles obtained from digital slides of the cancerous bodily tissue having the first type of cancer; e.g., digital slides of bodily tissue having adenocarcinoma.

At block 520, the computing device can receive second imaging data representing multiple second areas of cancerous bodily tissue having the second type of cancer (e.g., small cell carcinoma). The multiple second areas can correspond to tiles obtained from digital slides of the cancerous bodily tissue having the first type of cancer; e.g., digital slides of bodily tissue having small cell carcinoma of lung and/or digital slides of bodily tissue having small cell carcinoma of prostate.

At block 530, the computing device can generate first feature vectors for respective ones of the multiple areas. Generating each one of the first feature vectors includes determining multiple values defining respective tissue organization features. Each one of the first feature vectors is defined by a set of targeted features. As mentioned, those features can pertain to various descriptor categories, including fractal dimension, Gabor feature, binarized statistical image feature, and LPQ. The set of targeted features has n items. Thus, each one of the first feature vectors is an n-dimensional feature vector. As mentioned, n can be greater than 1000. In some cases, the tissue organization features can be embodied in histopathological features of neuroendocrine differentiation. In other cases, histopathological features of other forms of lineage plasticity also can be contemplated, in some embodiments.

At block 540, the computing device can generate second feature vectors for respective ones of the multiple second areas. Generating each one of the second feature vectors includes determining multiple values defining respective tissue organization features. Each one of second first feature vectors also is defined by the set of targeted features. Thus, such tissue organization features are the same as those considered in the generation of the first feature vectors.

At block 550, the computing device can generate, using the first feature vectors and the second feature vectors, multiple machine-learning models. Each one of the multiple machine-learning models can be generated to distinguish the first type of cancer from the second type of cancer. For example, targeted features (e.g., handcrafted features) that form the first feature vector can be used to train a first classification model, and targeted features (e.g., handcrafted features) that form the second feature vector can be used to a second classification model. As mentioned, to generate the multiple machine-learning models, the computing device can implement a bootstrapping approach or a cross-validation approach. Each one of the machine-learning models can be embodied in one of a SVM; an RF model; a multi-layer perceptron; GLMNET; or similar models.

The machine-learning models generated by implementing the example method 500 can be used to select a subset of the set of targeted features. Such a subset can define a feature vector that can be used to determine a score indicative of neuroendocrine differentiation in an image of a section of cancerous bodily tissue. As mentioned, that score can be referred to as NED score.

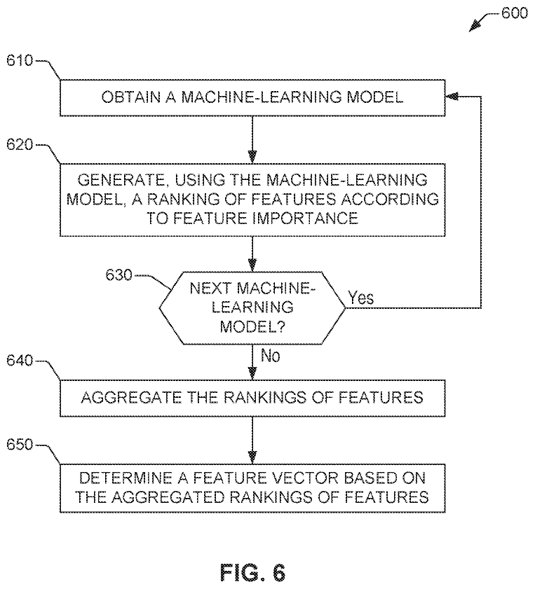

FIG. 6 illustrates an example of a method 600 for selecting features to define a feature vector that can be used to determine a NED score, in accordance with one or more embodiments of this disclosure. A computing device or a system of computing devices may implement the example method 600 in its entirety or in part. To that end, each one of the computing devices includes computing resources that may implement at least one of the blocks included in the example method 600. The computing resources can include, for example, CPUs, GPUs, TPUs, memory, disk space, incoming bandwidth, and/or outgoing bandwidth, interface(s) (such as I/O interfaces or APIs, or both); controller devices(s); power supplies; a combination of the foregoing; and/or similar resources. The computing resources also can include, for example, programming interface(s); an operating system; software for configuration and/or control of a virtualized environment; firmware; and similar resources. As an example, each of one or multiple ones of the computing devices can embody, or can host, the training subsystem 130 (FIG. 1).

At block 610, the computing device can obtain a machine-learning model. The machine-learning model can be obtained from a group of multiple machine-learning models trained to distinguish between a first type of cancer and a second type of cancer. Again, in one example, the first type of cancer is adenocarcinoma and the second type of cancer is small cell carcinoma. Such group of multiple machine-learning models can be generated by implementing the example method 500. Accordingly, in one example, the group of machine-learning models can be, or can include, the models 144 (FIG. 1).

At block 620, the computing device can generate, using the obtained machine-learning model, a ranking of features according to variable/feature importance. The ranking can be a list of features arranged in decreasing order of variable/feature importance. The feature importance can be obtained, at least partially, by applying the machine-learning model. The output of the application of the model can include the ranking.

At block 630, the computing device can determine if a next machine-learning model is to be obtained. That next machine-learning model can be obtained from the group of multiple machine-learning models trained to distinguish between a first type of cancer and a second type of cancer. Additionally, the next machine-learning model can be used to generate another ranking of features according to variable/feature importance. Accordingly, in response to ascertaining that the next machine-learning model is to be obtained, the flow of the example method 600 can be directed to block 610. In the alternative, in response to ascertaining that a next machine-learning model is not to be obtained, the flow of the example method 600 can be directed to block 640, where the computing device can aggregate the rankings of features, where each one of the ranking correspond to respective machine-learning models.

At block 650, the computing device can determine a feature vector based on the aggregated rankings of features. The feature vector is common to all machine-learning models used to generate rankings of features at block 620. Thus, a particular machine-learning model can be selected and can be applied to an instance of the feature vector in order to determine a NED score using an image of a section of cancerous bodily tissue having the first type of cancer (e.g., adenocarcinoma).

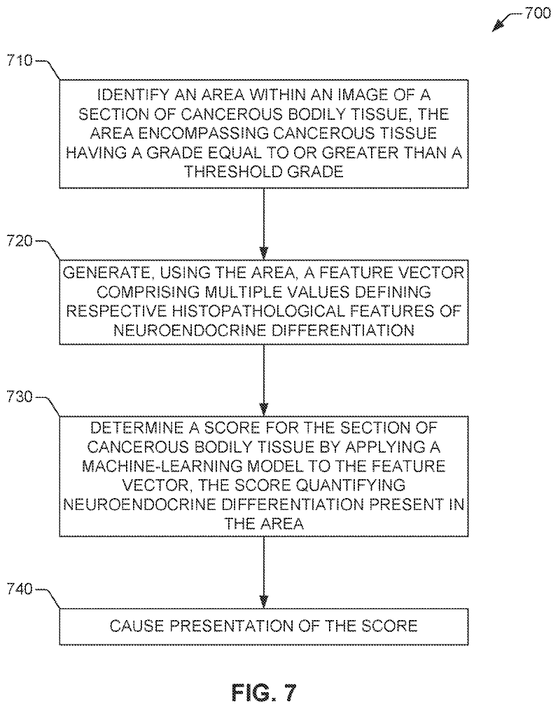

FIG. 7 illustrates an example of a method 700 for assessing an extent of neuroendocrine differentiation in a section of cancerous bodily tissue, in accordance with one or more embodiments of this disclosure. The cancerous bodily tissue has a defined type of cancer, such as adenocarcinoma. A computing device or a system of computing devices may implement the example method 700 in its entirety or in part. To that end, each one of the computing devices includes computing resources that may implement at least one of the blocks included in the example method 700. The computing resources can include, for example, CPUs, GPUs, TPUs, memory, disk space, incoming bandwidth, and/or outgoing bandwidth, interface(s) (such as I/O interfaces or APIs, or both); controller devices(s); power supplies; a combination of the foregoing; and/or similar resources. The computing resources also can include, for example, programming interface(s); an operating system; software for configuration and/or control of a virtualized environment; firmware; and similar resources. As an example, each of one or multiple ones of the computing devices can embody, or can host, the scoring subsystem 150 (FIG. 1) and can include, or can be functionally coupled to, the display device 160 (FIG. 1).

At block 710, the computing device can identify an area within an image of the section of the cancerous bodily tissue. The area encompasses cancerous tissue having a grade that is equal to or greater than a threshold grade. Identifying that area can include, for example, applying a cancer grading algorithm to the image of the section of cancerous bodily tissue, resulting in the grade, and comparing the resulting grade to the threshold grade. As noted herein, simply as an example, the grade can be one of the grade groups in the Gleason scale and the threshold grade can be grade group 4 in the Gleason scale. The area can correspond to a tile obtained from a digital slide of the cancerous bodily tissue.

At block 720, the computing device can generate, based on the area, a feature vector comprising multiple values defining respective tissue organization features. Those features can be, for example, histopathological features of neuroendocrine differentiation. The feature vector can be composed by tissue organization features that satisfy a variable/feature importance criterion. The feature vector can thus be defined or otherwise configured by implementing the example method 600 (FIG. 6), for example.

At block 730, the computing device can determine a score for the section of the cancerous bodily tissue by applying a machine-learning model to the feature vector. The score is the NED score described herein and, thus, quantifies neuroendocrine differentiation present in the area. As mentioned, the machine-learning model can be embodied in one of a SVM, a random forest model, a multi-layer perceptron, GLMNET, or similar models.

At block 740, the computing device can cause presentation of the score. Causing presentation of the score can include causing an output device to present the score, for example. The output device can be a display device, and causing the display device to present the score can include directing the display device to present a visual element identifying the score. In one example, the visual element can be presented at a user interface presented by the display device.

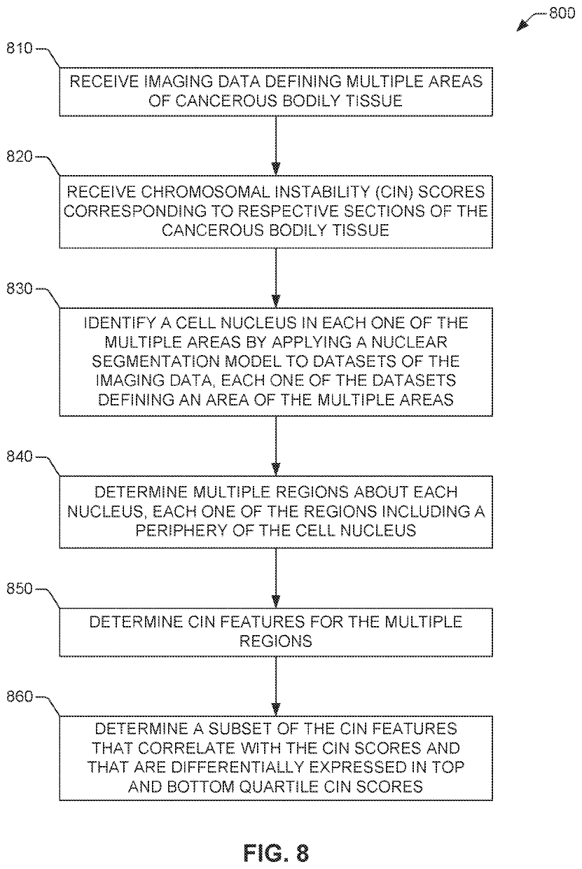

FIG. 8 illustrates an example of a method 800 for determining features of tissue organization of a section of cancerous bodily tissue, in accordance with one or more embodiments of this disclosure. The cancerous bodily tissue can have a particular type of cancer, such as adenocarcinoma. A computing device or a system of computing devices may implement the example method 800 in its entirety or in part. To that end, each one of the computing devices includes computing resources that may implement at least one of the blocks included in the example method 800. The computing resources can include, for example, CPUs, GPUs, TPUs, memory, disk space, incoming bandwidth, and/or outgoing bandwidth, interface(s) (such as I/O interfaces or APIs, or both); controller devices(s); power supplies; a combination of the foregoing; and/or similar resources. The computing resources also can include, for example, programming interface(s); an operating system; software for configuration and/or control of a virtualized environment; firmware; and similar resources. As an example, each of one or multiple ones of the computing devices can embody, or can host, the training subsystem 420 (FIG. 4A).

At block 810, the computing device can receive imaging data defining multiple areas of the cancerous bodily tissue. Each area of the multiple areas encompasses cancerous tissue having a grade equal to or greater than a threshold grade. The multiple areas can correspond to tiles obtained from digital slides of the cancerous bodily tissue.

At block 820, the computing device can receive CIN scores corresponding to respective sections of the cancerous bodily tissue. Each one of the CIN scores can be a score determined based on seven CIN genes identified using RNA sequencing data of sections of cancerous bodily tissue having a grade that is equal to or greater than the threshold grade. More specifically, the CIN scores can be determined for the cancerous tissue depicted in the multiple areas defined by the imaging data.

At block 830, the computing device can identify a cell nucleus in each one of the multiple areas by applying a nuclear segmentation model to datasets of the imaging data. Each one of the datasets defines an area of the multiple areas. In some cases, the nuclear segmentation model can be embodied in a mask R-CNN model.

At block 840, the computing device can determine multiple regions about each nucleus determined at block 830. Each one of the multiple regions includes a periphery of the cancer nucleus. Thus, each region of the multiple regions can be referred to as a perinuclear ring and corresponds to the perinuclear space of a nucleus. In some cases, three annular regions can be determined (see, for example, R1, R2, and R3 in FIG. 4B).

At block 850, the computing device can determine CIN features for the multiple regions. A defined set of targeted CIN features can be determined for each annular region. The CIN features can pertain to various descriptor categories that characterize morphology of a nucleus. Such categories include chromatin intensity, chromatin conformation, chromatin texture, shape, size, and nuclear proximity. That defined set can have m items. Thus, an m-dimensional feature vector can be generated for each annular region. In one example, m can be equal to 62.

At block 860, the computing device can determine a subset of the CIN features that correlate with the CIN scores and that are differentially expressed in top and bottom quartile CIN scores.

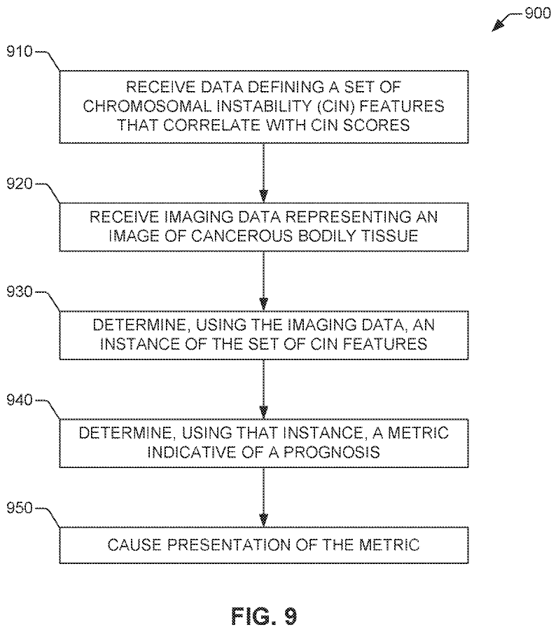

FIG. 9 illustrates an example of a method 900 for assessing survival prognosis of a subject hosting cancerous bodily tissue, in accordance with one or more embodiments of this disclosure. The cancerous bodily tissue can have a particular type of cancer, such as adenocarcinoma. A computing device or a system of computing devices may implement the example method 900 in its entirety or in part. To that end, each one of the computing devices includes computing resources that may implement at least one of the blocks included in the example method 900. As mentioned, the computing resources can include, for example, CPUs, GPUs, TPUs, memory, disk space, incoming bandwidth, and/or outgoing bandwidth, interface(s) (such as I/O interfaces or APIs, or both); controller devices(s); power supplies; a combination of the foregoing; and/or similar resources. The computing resources also can include, for example, programming interface(s); an operating system; software for configuration and/or control of a virtualized environment; firmware; and similar resources. As an example, each of one or multiple ones of the computing devices can embody, or can host, the scoring subsystem 150 (FIG. 4A) and can include, or can be functionally coupled to, the display device 160 (FIG. 4A).

At block 910, the computing device can receive data defining a set of CIN features that correlate with CIN scores. The set of CIN features can include one or multiple targeted descriptors. The targeted descriptors can include, for example, extranuclear DNA intensity and/or chromatin intensity; DNA conformation and/or chromatin conformation; DNA texture and/or chromatin texture; shape; size; and nuclear proximity. The set of CIN features can be generated by implementing the example method 800 (FIG. 8).

At block 920, the computing device can receive imaging data representing an image of cancerous bodily tissue. That image can be generated by imaging equipment that measures photoluminescence of the cancerous bodily tissue after that tissue has been stained with a dye (a mixture of hematoxylin and eosin, for example). The image can be generated with visible light.

At block 930, the computing device can determine, using the imaging data, an instance of the set of nuclear morphology features. Determining such an instance can include determining respective values of feature(s) in the set of nuclear morphology features.

At block 940, the computing device can determine a metric indicative of a prognosis of the particular type of cancer associated with the cancerous bodily tissue. The metric can be determined using the instance of the set of nuclear morphology features that has been determined at block 930.

At block 950, the computing device can cause presentation of the metric. Causing presentation of the score can include causing an output device to present indicia (digital or otherwise) representative of the metric. The output device can be a display device, and causing the display device to present such indicia can include directing the display device to present digital markings representative of the metric. The digital markings can be presented at a user interface presented by the display device.

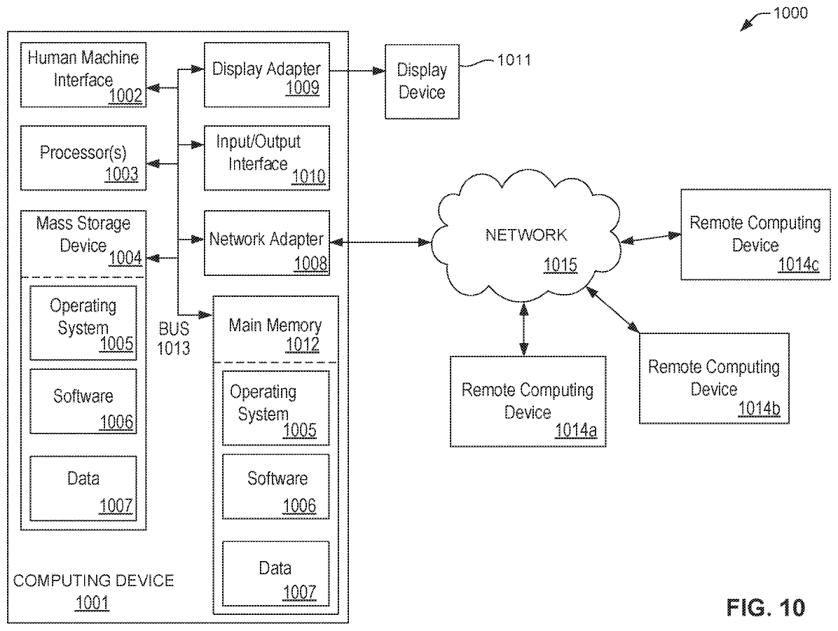

Computer-assisted point of care based on pathology image analysis in accordance with aspects described herein can be implemented on the computing system 1000 illustrated in FIG. 10 and described below. The computer-implemented methods and systems disclosed herein may utilize one or more computing devices to perform one or more functions in one or more locations. FIG. 10 is a block diagram depicting an example computing system 1000 for performing the disclosed methods and/or implementing the disclosed systems. The computing system 1000 is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment. The computing system 1000 shown in FIG. 10 may embody at least a portion of the example operating environment 100 (FIG. 1). In other cases, the computing system 1000 also can embody at least a portion of the example operating environment 400 (FIG. 4A). The computing system 1000 may implement the various functionalities described herein in connection with computer-assisted point of care based on pathology image analysis.

The computer-implemented methods and systems in accordance with this disclosure may be operational with numerous other general purpose or special purpose computing system environments or configurations. Examples of well-known computing systems, environments, and/or configurations that may be suitable for use with the systems and methods comprise, but are not limited to, personal computers, server computers, laptop devices, and multiprocessor systems. Additional examples comprise set-top boxes, programmable consumer electronics, network PCs, minicomputers, mainframe computers, distributed computing environments that comprise any of the above systems or devices, and the like.

The processing of the disclosed computer-implemented methods and systems may be performed by software components. The disclosed systems and computer-implemented methods may be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods may also be practiced in grid-based and distributed computing environments where tasks are performed by remote processing devices that are linked through a communications network. In a distributed computing environment, program modules may be located in both local and remote computer storage media including memory storage devices.

Further, the systems and computer-implemented methods disclosed herein may be implemented via a general-purpose computing device in the form of a computing device 1001. The components of the computing device 1001 may comprise one or more processors 1003, a system memory 1012, and a system bus 1013 that couples various system components including the one or more processors 1003 to the system memory 1012. The system may utilize parallel computing.

The system bus 1013 represents one or more of several possible types of bus structures, including a memory bus or memory controller, a peripheral bus, an accelerated graphics port, or local bus using any of a variety of bus architectures. The system bus 1013, and all buses specified in this description may also be implemented over a wired or wireless network connection and each of the subsystems, including the one or more processors 1003, a mass storage device 1004, an operating system 1005, software 1006, data 1007, a network adapter 1008, the system memory 1012, an Input/Output interface 1010, a display adapter 1009, a display device 1011, and a human-machine interface 1002, may be contained within one or more remote computing devices 1014a,b,c at physically separate locations, connected through buses of this form, in effect implementing a fully distributed system.

The computing device 1001 typically comprises a variety of computer-readable media. Exemplary readable media may be any available media that is accessible by the computing device 1001 and comprises, for example, both volatile and non-volatile media, removable and non-removable media. The main memory 1012 comprises computer readable media in the form of volatile memory, such as random access memory (RAM), and/or non-volatile memory, such as read only memory (ROM). The main memory 1012 typically contains data such as the data 1007 and/or program modules such as the operating system 1005 and the software 1006 that are immediately accessible to and/or are presently operated on by the one or more processors 1003. The operating system 1005 may be embodied in one of Windows operating system, Unix, or Linux, for example. In some cases, the software 1006 can include the training subsystem 130 (FIG. 1) and/or the training subsystem 420 (FIG. 4A). In addition, or in other cases where the computing device 1001 is deployed at point of care, the software 1006 can include the scoring subsystem 150. In those other cases, one or several of the remote computing devices 1014a,b,c can contain the data storage 140 (FIG. 1 and FIG. 4) and the computing device 1001 can obtain trained machine-learning models from at least one of such remote devices. Additionally, the display device 1011 can embody the display device 160 (FIG. 1).

In another aspect, the computing device 1001 may also comprise other removable/non-removable, volatile/non-volatile computer storage media. For example, FIG. 10 illustrates the mass storage device 1004 which may provide non-volatile storage of computer code, computer readable instructions, data structures, program modules, and other data for the computing device 1001. For example and not meant to be limiting, the mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules may be stored on the mass storage device 1004, including by way of example, the operating system 1005 and the software 1006. Each of the operating system 1005 and the software 1006 (or some combination thereof) may comprise elements of the programming and the software 1006. The data 1007 may also be stored on the mass storage device 1004. The data 1007 may be stored in any of one or more databases known in the art. Examples of such databases comprise, DB2®, Microsoft® Access, Microsoft® SQL Server, Oracle®, mySQL, PostgreSQL, and the like. The databases may be centralized or distributed across multiple systems.

In another aspect, the user may enter commands and information into the computing device 701 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices may be connected to the one or more processors 1003 via the human-machine interface 1002 that is coupled to the system bus 1013, but may be connected by other interface and bus structures, such as a parallel port, game port, an IEEE 1394 Port (also known as a Firewire port), a serial port, or a universal serial bus (USB).

In yet another aspect, the display device 1011 also can be connected to the system bus 1013 via an interface, such as the display adapter 1009. It is contemplated that the computing device 1001 may have more than one display adapter 1009 and the computing device 701 may have more than one display device 1011. For example, the display device 1011 may be a monitor, an LCD (Liquid Crystal Display), or a projector. In addition to the display device 711, other output peripheral devices may comprise components such as speakers (not shown) and a printer (not shown) which may be connected to the computing device 701 via the Input/Output interface 710. Any operation and/or result of the methods may be output in any form to an output device. Such output may be any form of visual representation, including, for example, textual, graphical, animation, audio, tactile, and the like. Although the display device 1011 is shown as being separate from the computing device 1001, the display device 1011 can be integrated into the computing device 1001 in some cases.

The computing device 1001 may operate in a networked environment using logical connections to one or more remote computing devices 1014a,b,c. For example, a remote computing device may be a personal computer, portable computer, smartphone, a server device, a router device, a network computer, a peer device or other common network node, and so on. Logical connections between the computing device 1001 and a remote computing device 1014a,b,c may be made via a network 1015, such as a LAN and/or a general WAN. Such network connections may be through the network adapter 1008. The network adapter 1008 may be implemented in both wired and wireless environments.

For purposes of illustration, application programs and other executable program components, such as the operating system 1005, are illustrated herein as discrete blocks, although it is recognized that such programs and components reside at various times in different storage components of the computing device 1001, and are executed by the one or more processors 1003 of the computer. An implementation of the software 1006 may be stored on or transmitted across some form of computer-readable media. Any of the disclosed methods may be performed by computer readable instructions embodied on computer-readable media. Computer-readable media may be any available media that may be accessed by a computer. By way of example and not meant to be limiting, computer-readable media may comprise “computer storage media” and “communications media.” “Computer storage media” comprise volatile and non-volatile, removable and non-removable media implemented in any methods or technology for storage of information such as computer-readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by a computer.

### Pathology Image Analysis

Convolutional neural networks (CNNs) are commonly used in histopathology. Because digital whole slide images (WSIs) in pathology are much larger than typical input sizes for CNNs, workflows typically first tile the WSI into many smaller patches. There are two main approaches for training classification models with WSIs: strong and weak supervision. Strong supervision uses labels for the individual tiles, which requires expert annotation at a high cost. Weak supervision applies multiple instance learning with slide level labels. Weakly supervised methods have become popular due to the ease of obtaining labels for learning directly from pathology reports. However, successful model training with weak learning requires thousands of WSIs, and strong supervision is still essential when a smaller number of WSIs are available for learning.

Expert annotation at the tile level is infeasible to obtain beyond a small number of WSIs. Semi-supervised learning (SSL) seeks to leverage unlabeled data to improve the accuracy of models when only a limited amount of labeled data is available. One of the recent trends in SSL, consistency regularization, has also found application in the classification of histopathology images. Teacher-student consistency has been used to supplement tile-level labels for quantifying prognostic features in colorectal cancer and in combination with weak supervision for Gleason grade classification in prostate cancer. The Mix-Match model has been tested on histology datasets with open-set noise. Weak/strong data transformation consistency (FixMatch) has been applied to detection of dysplasia of the esophagus. State-of-the-art SSL methods rely on enforcing prediction/representation consistency between various transformations of the data. Whereas consistency under model perturbations has been proposed, it is a less explored area. On the other hand, the co-training approach to SSL can provide excellent results when multiple views of each sample are available that meet the criteria of sufficiency (each view should be able to support accurate classification on its own) and conditional independence given the label of a sample.

Hematoxylin (H) and Eosin (E) are chemical stains that are used to highlight features of tissue architecture in formalin-fixed and paraffin-embedded tissue sections. H and E provide complementary information for pathologists. H is a basic chemical compound that binds negatively charged nucleotides in DNA and RNA to provide a blue color. In contrast, E is acidic and reacts with basic side chains of amino acids resulting in pink coloration. Whereas proteins bind to DNA and RNA lead to overlapping H and E staining in the cell nucleus and cytoplasm, the extracellular matrix and vascular structures supporting cancer cells interact primarily with E since they are devoid of DNA and RNA. In contrast to RGB channels that cannot easily be linked to a biological interpretation, H and E allow separation of nuclei versus cytoplasm and extracellular matrix. Therefore, H and E stains, when separated into their own channels, can provide two views that can, to a large extent, satisfy the co-training assumptions. A novel contrastive co-training with H and E views is also formulated. This approach is validated on a dataset of 53 WSIs from clear cell renal cell carcinoma (ccRCC) patients for histologic growth pattern (HGP) classification and of 45 WSIs from prostate cancer patients for cancer vs. benign gland classification. This approach was demonstrated to outperform state-of-the-art SSL methods. Additional experiments were performed to explain the suitability of H and E channels for co-training as opposed to RGB channels.

The approach includes separation of an H&E image in the RGB space into individual H and E stain channels using non-linear pixel-wise functions derived from dominant color profiles of each stain. The following approximate transformation between the two spaces is used:

$$[ \begin{matrix}H \\ E\end{matrix} ] = [ \begin{matrix}1.838 & 0.034 & - 0.76 \\ - 1.373 & 0.772 & 1.215\end{matrix} ] [ \begin{matrix}{log}_{10} 255 / R \\ {log}_{10} 255 / G \\ {log}_{10} 255 / B\end{matrix} ] . \qquad ( Eq . 1 )$$

The H and E channels are normalized to the range [0, 1] after the transformation.

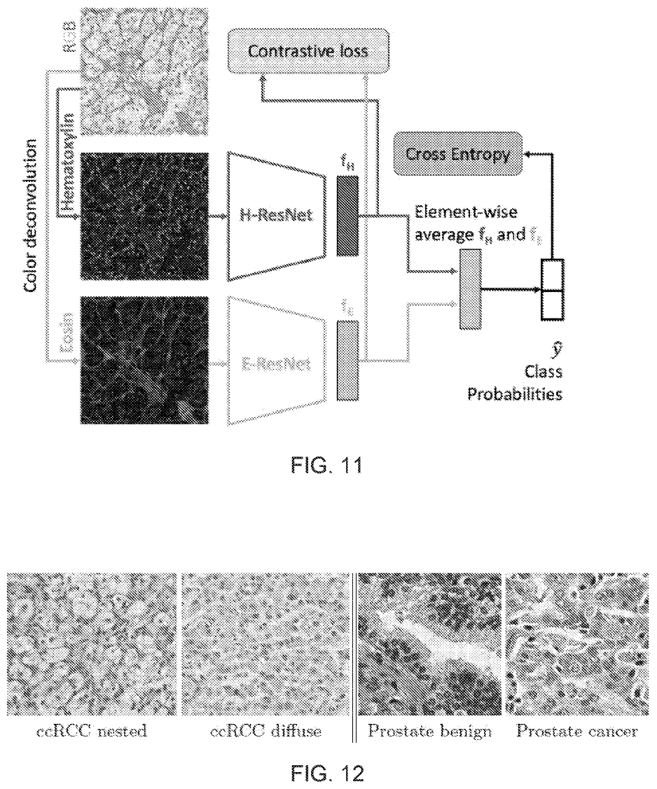

Two ResNet models (e.g., same architecture, separate parameters) are used for H and E channels, respectively (FIG. 11). Existing co-training methods enforce consistency of prediction between classifier outputs operating on different views of the data. The disadvantage of this approach is that the individual classifiers only make use of their respective views and are sub-optimal. Instead, the present disclosure uses a contrastive loss in the feature space to implement co-training and define a single classifier which uses a combined view by averaging the features from the two channels (FIG. 11). This approach is inspired by recent works that use contrastive learning to create a shared feature space between multimodal data. A contrastive loss is used to create a shared feature space between features extracted by the H and E networks. Let f_{H}(x) and f_{E}(x) denote the H and E features for input tile x, respectively. A triplet loss is used:

$${L}_{c . t .} ( {x}_{i} ) = max ( {‖ {f}_{H} ( {x}_{i} ) - {f}_{E} ( {x}_{i} ) ‖}_{2} - {‖ {f}_{H} ( {x}_{i} ) - {f}_{E} ( {x}_{k} ) ‖}_{2} + m , 0 ) , \qquad ( Eq . 2 )$$

where random k≠i, ∥a∥₂ denotes the L2 norm of vector a, and m is the margin hyperparameter. The triplet loss encourages (f_{H}, f_{E}) pairs from the same H&E tile xᵢ to be mapped closer together than (f_{H}>f_{E}) pairs from mismatched input tiles xᵢ and xₖ. Note that the output of the model is a linear+softmax layer applied to 0.5(f_{H}+f_{E}). Therefore, pushing the features f_{H} and f_{E} closer for the same tile also implicitly minimizes the difference between individual predictions, similar to co-training, if the final layer were applied to f_{H} and f_{E} alone.

Let L={xⱼ, yⱼ} denote the labeled training set where yᵢ is the label corresponding to input tile xᵢ. Let U={xᵢ} denote the unlabeled training set. The overall learning strategy combines supervised learning with cross-entropy on the labeled dataset with the triplet loss (Eq. 2) on the entire dataset:

$$ℒ = {\sum }_{{x}_{j} ∈ L} {y}_{j} log {\hat{y}}_{j} + λ {\sum }_{{x}_{i} ∈ L ⋃ U} {ℒ}_{c . t .} ( {x}_{i} ) , \qquad ( Eq . 3 )$$

where ŷⱼ denotes the output of the model for input xⱼ and λ is a hyperparameter controlling the relative contributions from the labeled and unlabeled losses.

### Example

### Datasets

Clear cell renal cell carcinoma (ccRCC). H&E slides from ccRCC patients were retrieved from the pathology archive and scanned at 40× magnification. HGPs in 53 WSIs were annotated by drawing polygons around them in QuIP by a GU-subspecialty trained pathologist. HGPs were divided into nested vs. diffuse (non-nested) histologic classes. Diffuse HGPs are associated with a higher risk of cancer recurrence and metastatic progression. Each WSI contains multiple polygons. Images were downsampled by a factor of 2× and a tile size of 400×400 pixels was chosen to capture the visual characteristics of the HGPs after discussion with pathologists (FIG. 12). Overlapping tiles were sampled by choosing a stride of 200 pixels. 3014 tiles with nested HGPs and 2566 tiles with diffuse HGPs were extracted from the annotated polygons. The WSIs were separated into training, validation and testing sets to ensure a realistic experimental setting. This separation resulted in 2116/1990 nested/diffuse tiles for training, 386/246 nested/diffuse tiles for validation and 512/330 nested/diffuse tiles for testing. The validation set was used for choosing hyperparameters as discussed below. The test set was for final model evaluation. Tiles from same patient were in the same set. For the SSL experiments, the annotated polygons from the training set were divided into 10 groups and randomly picked one group to draw labeled tiles from in each run of the experiments. This is a more realistic and challenging scenario than randomly choosing 10% of the training tiles as labeled data because tiles from the same polygon usually represent a smaller range of variations for learning.

Prostate Cancer. 6,992 benign gland images and 6,992 prostate cancer images were collected as a training set using the same process as with the ccRCC dataset. The tile size was chosen as 256×256, which is sufficient to characterize gland features. The validation and test sets are from the The Cancer Genome Atlas Program (TCGA). 477 benign and 472 cancer tiles were collected from 18 cases. In each experiment, 8 cases were randomly selected for validation and 10 cases for testing. Examples of prostate gland images are shown in FIG. 12. For SSL, the training images were randomly divided into 20 groups and one group was used as labeled data.

### Model Selection, Training and Hyperparameters

Considering the small number of training samples, ImageNet pretrained ResNet18 was selected for all experiments. ResNet is a state-of-the-art model which has better performance with less parameters. For models that use single channel inputs, i.e., the H and E CNN pathways in FIG. 11, the convolutional weights of the R, G and B channels in the first layer of ResNet18 were summed. The final layer of the ResNet18 was also changed for binary classification.

Color jittering, random rotation, crop to 256×256(ccRCC) or 224×224(prostate) pixels, random horizontal/vertical flip and color normalization were used as data augmentation. For validation and test tiles, center crop and color normalization were performed to follow the same data format as in training. For the co-training model, H and E channels have independent color jitters but the rest of the augmentations are common, e.g., the same random rotation angle was applied to the H and E channels from the same tile. The rationale for independent color jitters was that color variations due to the amount of H or E chemical tissue stains used are common in practice, which leads to independent brightness variations in these channels.

The Adam optimizer with an initial learning rate of 10⁻³ (100% label only) or 10⁻⁴ and a decaying learning rate was used. A batch size of 64 was used in the ccRCC dataset and 128 in the prostate cancer dataset. Hyperparameters in (3) were chosen as λ=0.2×p and m=40, where p is the percentage of training data used as labeled data. All hyperparameters were chosen to optimize accuracy over the validation set, including experiments on other state-of-the-art models. Batch normalization was applied to the features before computing the contrastive loss.

For comparison with other state-of-the-art SSL methods, consistency regularization, MixMatch and FixMatch were used. The same augmentations discussed above were used for the SSL experiments. All experiments were run for 250 epochs in ccRCC experiments and 100 epochs in prostate cancer dataset and chose the epoch with the best validation accuracy. Each experimental setting was run 5 times to calculate mean accuracy and standard deviation.

Python 3.7.11+Pytorch 1.9.0+torchvision 0.10.0+CUDA 10.2 were used on virtual environment and run on NVIDIA TITAN X and NVIDIA TITAN RTX. Python 3.9.0+Pytorch 1.7.1+torchvision 0.8.2+CUDA 11.0 were also used and run on NVIDIA RTX A6000. With batchsize fixed to 64, co-training experiment on ccRCC occupied around 5300 MB memory on GPU and needed 1.5-2.0 minutes for each epoch. The code is available at https://github.com/BzhangURU/Paper_2022_Co-training.

### Results

Proposed co-training with H and E views were compared to a baseline ResNet18 model that uses RGB H&E images as input, as well as other state-of-the-art SSL methods, such as MixMatch and FixMatch, considering they are already widely used in histopathology image analysis. The approaches were compared under two settings: using 100% of the available labeled tiles in training set for supervised learning and using only a subset (10% in ccRCC, 5% in prostate) of the available tiles for supervised learning. The proposed model also employed the unsupervised co-training loss with 100% of the training data (unlabeled) to set up an SSL method. Mean accuracy and standard deviation over 5 runs reported for all methods are shown in Table 1 for both datasets.

TABLE 1
Mean accuracy and standard deviations of different models for the test
sets in ccRCC and prostate experiments. Best performing model results
for the 100% and 10%/5% labeled data setting are shown in bold.

| ccRCC Model | Test Accuracy | Prostate Model | Test Accuracy |
|---|---|---|---|
| 100% label RGB ResNet | 84.8 ± 2.4% | 100% label RGB ResNet | 77.5 ± 2.5% |
| 100% label H/E co-train | 92.0 ± 2.6% | 100% label H/E co-train | 79.1 ± 2.0% |
| 10% label RGB ResNet | 76.9 ± 5.9% | 5% label RGB ResNet | 73.4 ± 1.0% |
| 10% label RGB consis | 86.8 ± 3.3% | 5% label RGB consis | 74.7 ± 1.3% |
| 10% label RGB MixMatch | 85.9 ± 5.7% | 5% label RGB MixMatch | 73.7 ± 5.0% |
| 10% label RGB FixMatch | 88.3 ± 3.8% | 5% label RGB FixMatch | 78.2 ± 3.8% |
| 10% label H/E co-train | 92.3 ± 2.1% | 5% label H/E co-train | 78.7 ± 1.9% |

It was noted that the contrastive co-training strategy improved test accuracy, by a large margin in the case of ccRCC, when 100% of the labeled data were used for supervised training (row 2 vs. 1, Table 1), which suggested it provided a strong regularization effect against overfitting. Note that training accuracy for the fully supervised RGB ResNet and H/E co-train models were 99.97±0.02% and 99.78±0.15%, respectively, in the ccRCC dataset. The same models achieve 98.34±0.64% and 98.32±0.55% training accuracy in prostate cancer. The fact that test accuracies on prostate cancer are lower than on ccRCC for all models is likely due to domain shift. In ccRCC dataset, all training, validation and test sets come from our institution. While in prostate cancer dataset, only training set comes our institution, the validation and test set come from TCGA dataset. Another possible reason is the fact that sometimes the gland size in prostate cancer is much smaller than the size of tiles, which could carry much less distinguishable features.

As expected, the proposed co-training strategy significantly outperformed the baseline approach (row 7 vs. 3, Table 1) under the limited labeled data setting. Consistency regularization based SSL methods significantly improve the accuracy of RGB ResNet baseline when a limited amount of training data is available (rows 4-6 vs. 3, Table 1). In line with results from computer vision, FixMatch even surpassed the baseline model trained with the entire labeled dataset. However, this disclosed method outperformed all other SSL methods compared against including FixMatch for both datasets. It is noted that hyperparameters for all SSL methods were independently fine-tuned to obtain the best validation accuracy. Finally, contrastive co-training was able to reach the same accuracy levels independent of the amount of labeled data that was used for supervised training (rows 2 and 7, Table 1).

### Co-Training View Analysis

The suitability of the H and E channels for co-training in the context of the ccRCC dataset was further studied. First, it was explored whether the H and E channels are sufficient on their own to provide a basis for accurate classification in a supervised setting. Models that only use the H or only use the E channel as input were trained. The 100% labeled results in Table 2 show that both channels carry sufficient information for the classification problem at hand. This is especially true for the E-channel, which is particularly informative for the nested vs. diffuse classification task. However, as expected, the accuracy for both channels drops significantly when the labeled data is limited.

TABLE 2
H-only and E-only models test accuracy for the ccRCC dataset.

| Model | Accuracy | Model | Accuracy |
|---|---|---|---|
| 100% label H ResNet | 79.4 ± 3.7% | 10% label H ResNet | 73.5 ± 4.0% |
| 100% label E ResNet | 94.0 ± 1.4% | 10% label E ResNet | 82.3 ± 7.0% |

Next explored was whether the H and E channels are better suited for co-training than R, G and B channels due to a higher degree of independence. An image-to-image regression model was trained using the U-Net architecture between various channels, e.g., predicting the E-channel from the H-channel of the same tile. The final layer of the U-Net architecture was chosen to be linear, and the mean square error function was used for training. Table 3 reports the coefficient of determination (R²) achieved for various input/output channel combinations. It was observed that the H and E channels are harder to predict from each other (lower R²) compared to the R, G and B channels, hence demonstrating a higher degree of independence and suitability for co-training.

TABLE 3
Coefficient of determination(R2) of image mapping between various
channels on ccRCC validation set at epoch with the lowest MSE.

| Experiments | R² Value | Experiments | R² Value |
|---|---|---|---|
| H ⇒ E | 0.5223 | E ⇒ H | 0.4613 |
| R ⇒ G | 0.8464 | G ⇒ R | 0.7833 |
| R ⇒ B | 0.8207 | B ⇒ R | 0.7713 |
| G ⇒ B | 0.8522 | B ⇒ G | 0.8824 |

### Ablation Studies

Ablation studies were also conducted to separately analyze the role of the contrastive loss and the H and E channel selection in terms of classification accuracy on ccRCC. Omitting the contrastive loss from training while using the H and E channel inputs lowered the accuracy from 92.0±2.6% to 84.7±5.2% for 100% labeled data and from 92.3±2.1% to 78.7±8.0% for 10% labeled data. In the next ablation experiment, various pairs from the RGB channels were used as the basis for this co-training method, and compared with ResNet using the same pair as input, e.g., using only the R and B channels to form 2-channel images as input for ResNet. Results are reported in Table 4. Unlike the H and E models, it was observed that the results are approximately the same, which was expected considering the higher level of dependence among RGB channels shown above. These observations suggest that the benefit of the model is due to the contrastive co-training loss applied to the H and E view inputs rather than simply due to the change in the input space or the contrastive loss individually.

TABLE 4
Ablation study on ccRCC. Test set accuracy of ResNet and co-training models
taking only 2 channels from RGB as input with 10% labeled data in training.

| Model | Accuracy | Model | Accuracy | Model | Accuracy |
|---|---|---|---|---|---|
| RB ResNet | 77.5 ± 6.6% | RG ResNet | 80.2 ± 6.4% | GB ResNet | 78.4 ± 9.7% |
| R/B co-train | 78.2 ± 4.5% | R/G co-train | 79.8 ± 5.6% | G/B co-train | 76.6 ± 7.3% |

It is to be understood that the methods and systems described here are not limited to specific operations, processes, components, or structure described, or to the order or particular combination of such operations or components as described. It is also to be understood that the terminology used herein is for the purpose of describing example embodiments only and is not intended to be restrictive or limiting.

Throughout the specification and claims of this disclosure, the following words have the meaning that is set forth: “comprise” and variations of the word, such as “comprising” and “comprises,” mean including but not limited to, and are not intended to exclude, for example, other additives, components, integers, or operations. “Include” and variations of the word, such as “including” are not intended to mean something that is restricted or limited to what is indicated as being included, or to exclude what is not indicated. “May” means something that is permissive but not restrictive or limiting. “Optional” or “optionally” means something that may or may not be included without changing the result or what is being described. “Prefer” and variations of the word such as “preferred” or “preferably” mean something that is exemplary and more ideal, but not required. “Such as” means something that serves simply as an example.

Operations and components described herein as being used to perform the disclosed methods and construct the disclosed systems are illustrative unless the context clearly dictates otherwise. It is to be understood that when combinations, subsets, interactions, groups, etc. of these operations and components are disclosed, that while specific reference of each various individual and collective combinations and permutation of these may not be explicitly disclosed, each is specifically contemplated and described herein, for all methods and systems. This applies to all aspects of this application including, but not limited to, operations in disclosed methods and/or the components disclosed in the systems. Thus, if there are a variety of additional operations that may be performed or components that may be added, it is understood that each of these additional operations may be performed and components added with any specific embodiment or combination of embodiments of the disclosed systems and methods.

Embodiments of this disclosure may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof, whether internal, networked, or cloud-based.

Embodiments of this disclosure have been described with reference to diagrams, flowcharts, and other illustrations of computer-implemented methods, systems, apparatuses, and computer program products. Each block of the block diagrams and flowchart illustrations, and combinations of blocks in the block diagrams and flowchart illustrations, respectively, may be implemented by processor-accessible instructions. Such instructions may include, for example, computer program instructions (e.g., processor-readable and/or processor-executable instructions). The processor-accessible instructions may be built (e.g., linked and compiled) and retained in processor-executable form in one or multiple memory devices or one or many other processor-accessible non-transitory storage media. These computer program instructions (built or otherwise) may be loaded onto a general-purpose computer, special purpose computer, or other programmable data processing apparatus to produce a machine. The loaded computer program instructions may be accessed and executed by one or multiple processors or other types of processing circuitry. In response to execution, the loaded computer program instructions provide the functionality described in connection with flowchart blocks (individually or in a particular combination) or blocks in block diagrams (individually or in a particular combination). Thus, such instructions which execute on the computer or other programmable data processing apparatus create a means for implementing the functions specified in the flowchart blocks (individually or in a particular combination) or blocks in block diagrams (individually or in a particular combination).

These computer program instructions may also be stored in a computer-readable memory that may direct a computer or other programmable data processing apparatus to function in a particular manner, such that the instructions stored in the computer-readable memory produce an article of manufacture including processor-accessible instruction (e.g., processor-readable instructions and/or processor-executable instructions) to implement the function specified in the flowchart blocks (individually or in a particular combination) or blocks in block diagrams (individually or in a particular combination). The computer program instructions (built or otherwise) may also be loaded onto a computer or other programmable data processing apparatus to cause a series of operations to be performed on the computer or other programmable apparatus to produce a computer-implemented process. The series of operations may be performed in response to execution by one or more processor or other types of processing circuitry. Thus, such instructions that execute on the computer or other programmable apparatus provide operations for implementing the functions specified in the flowchart blocks (individually or in a particular combination) or blocks in block diagrams (individually or in a particular combination).

Accordingly, blocks of the block diagrams and flowchart diagrams support combinations of means for performing the specified functions in connection with such diagrams and/or flowchart illustrations, combinations of operations for performing the specified functions and program instruction means for performing the specified functions. Each block of the block diagrams and flowchart illustrations, and combinations of blocks in the block diagrams and flowchart illustrations, may be implemented by special purpose hardware-based computer systems that perform the specified functions or operations, or combinations of special purpose hardware and computer instructions.

Features utilized in various embodiments of this disclosure can be targeted features (for example, handcrafted features) that are directly extracted from image tiles. The features are not learned by the computer. In some cases, the features may not be generated by neural networks and may not rely on artificial intelligence (AI) framework to be defined. The methods and systems using targeted feature values to predict patient stage or prognosis may employ artificial intelligence techniques such as machine learning and iterative learning. Examples of such techniques include, but are not limited to, expert systems, case-based reasoning, Bayesian networks, behavior-based AI, neural networks, fuzzy systems, evolutionary computation (e.g., genetic algorithms), swarm intelligence (e.g., ant algorithms), and hybrid intelligent systems (e.g., expert inference rules generated through a neural network or production rules from statistical learning).

While the computer-implemented methods, apparatuses, devices, and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its operations be performed in a specific order. Accordingly, where a method claim does not actually recite an order to be followed by its operations or it is not otherwise specifically stated in the claims or descriptions that the operations are to be limited to a specific order, it is in no way intended that an order be inferred, in any respect. This holds for any possible non-express basis for interpretation, including: matters of logic with respect to arrangement of operations or operational flow; plain meaning derived from grammatical organization or punctuation; the number or type of embodiments described in the specification.

It will be apparent to those skilled in the art that various modifications and variations may be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A computer-implemented method, comprising:
identifying, by a computing device comprising at least one processor, an area within an image of a section of cancerous bodily tissue, the area encompassing cancerous tissue having a grade that is greater than or equal to a threshold grade;
generating, by the computing device, a feature vector within the area comprising multiple values defining respective targeted histopathologic features of neuroendocrine differentiation; and
determining a score for the section of cancerous bodily tissue by applying a machine-learning model to the feature vector, the score representing an amount of neuroendocrine differentiation present in the area.

2. The computer-implemented method of claim 1, further comprising causing a device to present the score.

3. The computer-implemented method of claim 1, wherein the grade is one of the grade groups in the Gleason scale, and wherein the threshold grade is a particular high grade in the Gleason scale.

4. The computer-implemented method of claim 1, wherein a first feature of the respective targeted histopathologic features of neuroendocrine differentiation pertains to one of the following categories: fractal dimension, Gabor, binarized statistical image, or local phase quantization (LPQ).

5. The computer-implemented method of claim 1, further comprising accessing, by the computing device, imaging data representing the image of the section of cancerous bodily tissue stained with hematoxylin and eosin, wherein the image is a regular pathology slide for microscopic diagnosis.

6. The computer-implemented method of claim 1, wherein the identifying comprises applying, by the computing system, a cancer grading algorithm to the image of the section of cancerous bodily tissue, resulting in the grade.

7. A computing device configured to perform one or more of the methods of claims 1 to 6.

8. A computing system, comprising:
at least one processor; and
at least one memory device storing processor-executable instructions that, in response to execution by the at least one processor, cause the computing system to perform one or more of the methods of claims 1 to 6.

9. At least one computer-readable medium having processor-executable instructions encoded thereon that, in response to execution, cause one or more computing devices to perform one or more of the methods of claims 1 to 6.

10. A computer-implemented method, comprising, receiving, by a computing device, data defining a set of nuclear morphology features that correlate with chromosomal instability (CIN) scores;
receiving, by the computing device, image data representing an image of cancerous bodily tissue;
determining, using the image data, an instance of the set of nuclear morphology features, the instance representing an extent of chromosomal instability inside a cell nucleus within the cancerous bodily tissue;
determining, using the instance, a metric indicative of a stage or a prognosis; and
causing a device to present the metric.

11. The computer-implemented method of claim 10, wherein a first feature of the set of nuclear morphology features pertains to one of the following descriptor categories: chromatin intensity, chromatin conformation, chromatin texture, shape, size, and nuclear proximity.

12. The computer-implemented method of claim 11, wherein each one of the CIN scores represents an amount of chromosomal instability inside the cell nucleus as measured by a gene expression panel.

13. A computing device configured to perform the method of any one of claims 10 to 12.

14. A computing system, comprising:
at least one processor; and
at least one memory device storing processor-executable instructions that, in response to execution by the at least one processor, cause the computing system to perform the method of any one of claims 10 to 12.

15. At least one non-transitory computer-readable storage medium having processor-executable instructions encoded thereon that, in response to execution, cause one or more computing devices to perform the method of any one of claims 10 to 12.

16. A computer-implemented method for pathology image classification, the method comprising:
receiving an image of a cancerous tissue sample for a patient;
generating a feature vector within an area on the image, wherein the feature vector includes multiple values defining respective targeted histopathologic features of neuroendocrine differentiation;
determining a metric for the area of cancerous tissue sample on the image by processing the feature vector through a model trained to distinguish tiles of non-metastatic (M0) adenocarcinoma from tiles of small cell carcinoma (SC), and wherein the metric represents an amount of neuroendocrine differentiation present in the area; and
providing the metric for further processing or to a user interface of a computer device.

17. The method of claim 16, wherein the model is trained for pathology image classification using a co-training approach leveraging deconvolution of an Hematoxylin (H) and Eosin (E) (H&E) image into individual H and E stains.

18. The method of claim 16, further comprising:
determining a prognosis of the patient and likelihood of treatment resistance of the area based on the metric and a threshold value.

19. A computing system, comprising:
at least one processor; and
at least one memory device storing processor-executable instructions that, in response to execution by the at least one processor, cause the computing system to perform one or more of the methods of claims 16 to 18.

20. At least one computer-readable medium having processor-executable instructions encoded thereon that, in response to execution, cause one or more computing devices to perform one or more of the methods of claims 16 to 18.
